(19) United States
(12) Patent Application Publication
(10) Pub. No.: US 2015/0361504 A1
(43) Pub. Date: Dec. 17, 2015

(54) BIOMARKER FOR PREDICTING RESPONSE OF CLL TO TREATMENT WITH A BTK INHIBITOR

(71) Applicant: Pharmacyclics LLC, Sunnyvale, CA (US)

(72) Inventors: John C. BYRD, Columbus, OH (US); Daphne GUINN, Columbus, OH (US); Amy JOHNSON, Dublin, OH (US)

(21) Appl. No.: 14/738,617

(22) Filed: Jun. 12, 2015

Related U.S. Application Data
(60) Provisional application No. 62/012,204, filed on Jun. 13, 2014.

(51) Int. Cl. C12Q 1/68; A61K 31/519

(52) U.S. Cl. CPC C12Q 1/6886; A61K 31/519; C12Q 2600/178; C12Q 2600/106

(57) ABSTRACT

Disclosed herein are methods for treating an individual diagnosed with a solid tumor or a hematological malignancy, such as chronic lymphocytic leukemia (CLL), for treatment with a Bruton's tyrosine kinase (BTK) inhibitor (e.g., ibrutinib) based on the expression level of miR-155. Also disclosed herein are methods for assessing whether an individual having a solid tumor or a hematological malignancy such as chronic lymphocytic leukemia (CLL) is responsive or likely to be responsive to therapy with a BTK inhibitor (e.g., ibrutinib). Further disclosed herein are methods of monitoring whether an individual receiving a BTK inhibitor (e.g., ibrutinib) for treatment of a solid tumor or a hematological malignancy such as chronic lymphocytic leukemia (CLL) has relapsed or is likely to have a relapse to therapy. Also disclosed herein are methods of selecting an individual having a solid tumor or a hematological malignancy such as chronic lymphocytic leukemia (CLL) for therapy with a BTK inhibitor (e.g., ibrutinib).

### RELATED APPLICATIONS

The present application claims the benefit of priority from U.S. Provisional Application No. 62/012,204, filed Jun. 13, 2014, which is herein incorporated by reference in its entirety.

### BACKGROUND OF THE INVENTION

Chronic lymphocytic leukemia (CLL) is generally considered an incurable disease and occurs commonly in elderly patients. CLL is a heterogeneous disease characterized as either aggressive or indolent, and these varied clinical courses correlate with several biologic markers of prognosis.

### SUMMARY OF THE INVENTION

Disclosed herein, in certain embodiments, is a method of assessing whether an individual having chronic lymphocytic leukemia (CLL) is responsive or likely to be responsive to therapy with ibrutinib, comprising: (a) administering a treatment comprising ibrutinib; (b) determining an expression level of miR-155 in a sample from the individual following administration of the treatment; and (c) characterizing the individual as responsive or likely to be responsive to therapy if the individual shows a decrease in the expression level of miR-155 relative to a control. Further disclosed herein, in certain embodiments, is a method of monitoring whether an individual receiving ibrutinib for treatment of chronic lymphocytic leukemia (CLL) has relapsed or is likely to have a relapse to therapy, comprising: (a) administering a treatment comprising ibrutinib; (b) determining an expression level of miR-155 in a sample from the individual following administration of the treatment; and (c) characterizing the individual as relapsed or likely to have a relapse to therapy if the individual does not show a decrease in the expression level of miR-155 relative to a control. In some embodiments, the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the control is the expression level of miR-155 in the individual prior to treatment with ibrutinib. In some embodiments, the expression level of miR-155 is measured on day 1, 2, 3, 4, 5, 6, 7, 8, 9, 10, 12, 14, 16, 18, 20, 25, 29, or more following treatment with ibrutinib. In some embodiments, CLL is characterized by cytogenetic abnormalities. In some embodiments, the cytogenetic abnormalities comprise del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, or a combination thereof. In some embodiments, CLL is a refractory CLL. In some embodiments, CLL is a relapsed CLL. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the methods further comprise detection of the nucleic acid using a microarray. In some embodiments, the methods further comprise amplification of the nucleic acid. In some embodiments, the amplification is a polymerase chain reaction. In some embodiments, the treatment further comprises a second anticancer therapy. In some embodiments, the second anticancer therapy is a chemotherapeutic agent. In some embodiments, the chemotherapeutic agent is selected from among ofatumumab, rituximab, fludarabine, or a combination thereof. In some embodiments, the chemotherapeutic agent is ofatumumab. In some embodiments, the individual has received previous anticancer therapy. In some embodiments, the individual has not received previous anticancer therapy. In some embodiments, ibrutinib is administered at a dosage of about 40 mg/day to about 1000 mg/day. In some embodiments, ibrutinib is administered orally. In some embodiments, ibrutinib is administered once a day, two times per day, three times per day, four times per day, or five times per day.

Disclosed herein, in certain embodiments, is a method of treating an individual having chronic lymphocytic leukemia (CLL), comprising: (a) administering a treatment comprising ibrutinib; (b) determining an expression level of miR-155 in a sample from the individual following administration of the treatment; and (c) continuing the treatment if the expression level of miR-155 is decreased by a predetermined amount relative to the expression level of miR-155 prior to the treatment. Further disclosed herein, in certain embodiments, is a method of treating an individual having chronic lymphocytic leukemia (CLL), comprising: (a) administering a treatment comprising ibrutinib; (b) determining an expression level of miR-155 in a sample from the individual following administration of the treatment; and (c) discontinuing the treatment if the expression level of miR-155 is not decreased by a predetermined amount relative to the expression level of miR-155 prior to the treatment. In some embodiments, the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. Also disclosed herein, in certain embodiments, is a method of optimizing the treatment of chronic lymphocytic leukemia (CLL) in an individual in need thereof, comprising: (a) administering a treatment comprising ibrutinib; (b) determining an expression level of miR-155 in a sample from the individual following administration of the treatment; and (c) modifying the treatment based on the expression level of miR-155 relative to a control. In some embodiments, the control is the expression level of miR-155 in the individual prior to treatment with ibrutinib. In some embodiments, the expression level of miR-155 is measured on day 1, 2, 3, 4, 5, 6, 7, 8, 9, 10, 12, 14, 16, 18, 20, 25, 29, or more following treatment with ibrutinib. In some embodiments, CLL is characterized by cytogenetic abnormalities. In some embodiments, the cytogenetic abnormalities comprise del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, or a combination thereof. In some embodiments, CLL is a relapsed or refractory CLL. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the methods further comprise detection of the nucleic acid using a microarray. In some embodiments, the methods further comprise amplification of the nucleic acid. In some embodiments, the amplification is a polymerase chain reaction. In some embodiments, the treatment further comprises a second anticancer therapy. In some embodiments, the second anticancer therapy is a chemotherapeutic agent. In some embodiments, the chemotherapeutic agent is selected from among ofatumumab, rituximab, fludarabine, or a combination thereof. In some embodiments, the chemotherapeutic agent is ofatumumab. In some embodiments, the individual has received previous anticancer therapy. In some embodiments, the individual has not received previous anticancer therapy. In some embodiments, ibrutinib is administered at a dosage of about 40 mg/day to about 1000 mg/day. In some embodiments, ibrutinib is administered orally. In some embodiments, ibrutinib is administered once a day, two times per day, three times per day, four times per day, or five times per day.

Disclosed herein, in certain embodiments, is a method of selecting an individual having chronic lymphocytic leukemia (CLL) for therapy with ibrutinib, comprising: (a) measuring the expression level of miR-155 in a sample from the individual; (b) comparing the expression level of miR-155 with a reference level; and (c) characterizing the individual as a candidate for therapy with ibrutinib if the individual has an elevated level of miR-155 compared to the reference level. In some embodiments, the elevated level of miR-155 is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher in the expression of miR-155. In some embodiments, the reference level is the expression level of miR-155 in an individual who does not have CLL. In some embodiments, the expression level of miR-155 is measured on day 1, 2, 3, 4, 5, 6, 7, 8, 9, 10, 12, 14, 16, 18, 20, 25, 29, or more following treatment with ibrutinib. In some embodiments, CLL is characterized by cytogenetic abnormalities. In some embodiments, the cytogenetic abnormalities comprise del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, or a combination thereof. In some embodiments, CLL is a relapsed or refractory CLL. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the method further comprises detection of the nucleic acid using a microarray. In some embodiments, the method further comprises amplification of the nucleic acid. In some embodiments, the amplification is a polymerase chain reaction. In some embodiments, the treatment further comprises a second anticancer therapy. In some embodiments, the second anticancer therapy is a chemotherapeutic agent. In some embodiments, the chemotherapeutic agent is selected from among ofatumumab, rituximab, fludarabine, or a combination thereof. In some embodiments, the chemotherapeutic agent is ofatumumab. In some embodiments, the individual has received previous anticancer therapy. In some embodiments, the individual has not received previous anticancer therapy.

Disclosed herein, in certain embodiments, is a method of assessing whether an individual having a hematological malignancy (e.g., a B-cell or a T-cell malignancy) is responsive or likely to be responsive to therapy with a BTK inhibitor (e.g., an irreversible BTK inhibitor such as ibrutinib), comprising: (a) administering a treatment comprising the BTK inhibitor; (b) determining an expression level of miR-155 in a sample from the individual following administration of the treatment; and (c) characterizing the individual as responsive or likely to be responsive to therapy if the individual shows a decrease in the expression level of miR-155 relative to a control. Further disclosed herein, in certain embodiments, is a method of monitoring whether an individual receiving a BTK inhibitor (e.g., an irreversible BTK inhibitor such as ibrutinib) for treatment with a hematological malignancy (e.g., a B-cell or a T-cell malignancy) has relapsed or is likely to have a relapse to therapy, comprising: (a) administering a treatment comprising the BTK inhibitor; (b) determining an expression level of miR-155 in a sample from the individual following administration of the treatment; and (c) characterizing the individual as relapsed or likely to have a relapse to therapy if the individual does not show a decrease in the expression level of miR-155 relative to a control. In some embodiments, the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with the BTK inhibitor. In some embodiments, the control is the expression level of miR-155 in the individual prior to treatment with the BTK inhibitor. In some embodiments, the expression level of miR-155 is measured on day 1, 2, 3, 4, 5, 6, 7, 8, 9, 10, 12, 14, 16, 18, 20, 25, 29, or more following treatment with the BTK inhibitor. In some embodiments, hematological malignancy is characterized by cytogenetic abnormalities. In some embodiments, hematological malignancy is a refractory hematological malignancy. In some embodiments, hematological malignancy is a relapsed hematological malignancy. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the methods further comprise detection of the nucleic acid using a microarray. In some embodiments, the methods further comprise amplification of the nucleic acid. In some embodiments, the amplification is a polymerase chain reaction. In some embodiments, the treatment further comprises a second anticancer therapy. In some embodiments, the second anticancer therapy is a chemotherapeutic agent. In some embodiments, the chemotherapeutic agent is selected from among ofatumumab, rituximab, fludarabine, or a combination thereof. In some embodiments, the chemotherapeutic agent is ofatumumab. In some embodiments, the individual has received previous anticancer therapy. In some embodiments, the individual has not received previous anticancer therapy. In some embodiments, the BTK inhibitor is administered at a dosage of about 40 mg/day to about 1000 mg/day. In some embodiments, the BTK inhibitor is administered orally. In some embodiments, the BTK inhibitor is administered once a day, two times per day, three times per day, four times per day, or five times per day.

Disclosed herein, in certain embodiments, is a method of treating an individual having a hematological malignancy (e.g., a B-cell or a T-cell malignancy), comprising: (a) administering a treatment comprising a BTK inhibitor (e.g., an irreversible BTK inhibitor such as ibrutinib); (b) determining an expression level of miR-155 in a sample from the individual following administration of the treatment; and (c) continuing the treatment if the expression level of miR-155 is decreased by a predetermined amount relative to the expression level of miR-155 prior to the treatment. Further disclosed herein, in certain embodiments, is a method of treating an individual having a hematological malignancy (e.g., a B-cell or a T-cell malignancy), comprising: (a) administering a treatment comprising a BTK inhibitor (e.g., an irreversible BTK inhibitor such as ibrutinib); (b) determining an expression level of miR-155 in a sample from the individual following administration of the treatment; and (c) discontinuing the treatment if the expression level of miR-155 is not decreased by a predetermined amount relative to the expression level of miR-155 prior to the treatment. In some embodiments, the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with the BTK inhibitor. Also disclosed herein, in certain embodiments, is a method of optimizing the treatment of a hematological malignancy (e.g., a B-cell or a T-cell malignancy) in an individual in need thereof, comprising: (a) administering a treatment comprising a BTK inhibitor (e.g., an irreversible BTK inhibitor such as ibrutinib); (b) determining an expression level of miR-155 in a sample from the individual following administration of the treatment; and (c) modifying the treatment based on the expression level of miR-155 relative to a control. In some embodiments, the control is the expression level of miR-155 in the individual prior to treatment with the BTK inhibitor. In some embodiments, the expression level of miR-155 is measured on day 1, 2, 3, 4, 5, 6, 7, 8, 9, 10, 12, 14, 16, 18, 20, 25, 29, or more following treatment with the BTK inhibitor. In some embodiments, hematological malignancy is characterized by cytogenetic abnormalities. In some embodiments, hematological malignancy is a relapsed or refractory hematological malignancy. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the methods further comprise detection of the nucleic acid using a microarray. In some embodiments, the methods further comprise amplification of the nucleic acid. In some embodiments, the amplification is a polymerase chain reaction. In some embodiments, the treatment further comprises a second anticancer therapy. In some embodiments, the second anticancer therapy is a chemotherapeutic agent. In some embodiments, the chemotherapeutic agent is selected from among ofatumumab, rituximab, fludarabine, or a combination thereof. In some embodiments, the chemotherapeutic agent is ofatumumab. In some embodiments, the individual has received previous anticancer therapy. In some embodiments, the individual has not received previous anticancer therapy. In some embodiments, the BTK inhibitor is administered at a dosage of about 40 mg/day to about 1000 mg/day. In some embodiments, the BTK inhibitor is administered orally. In some embodiments, the BTK inhibitor is administered once a day, two times per day, three times per day, four times per day, or five times per day.

Disclosed herein, in certain embodiments, is a method of selecting an individual having a hematological malignancy (e.g., a B-cell or a T-cell malignancy) for therapy with a BTK inhibitor (e.g., an irreversible BTK inhibitor such as ibrutinib), comprising: (a) measuring the expression level of miR-155 in a sample from the individual; (b) comparing the expression level of miR-155 with a reference level; and (c) characterizing the individual as a candidate for therapy with the BTK inhibitor if the individual has an elevated level of miR-155 compared to the reference level. In some embodiments, the elevated level of miR-155 is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher in the expression of miR-155. In some embodiments, the reference level is the expression level of miR-155 in an individual who does not have a hematological malignancy. In some embodiments, the hematological malignancy is characterized by cytogenetic abnormalities. In some embodiments, CLL is a relapsed or refractory CLL. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the method further comprises detection of the nucleic acid using a microarray. In some embodiments, the method further comprises amplification of the nucleic acid. In some embodiments, the amplification is a polymerase chain reaction. In some embodiments, the treatment further comprises a second anticancer therapy. In some embodiments, the second anticancer therapy is a chemotherapeutic agent. In some embodiments, the chemotherapeutic agent is selected from among ofatumumab, rituximab, fludarabine, or a combination thereof. In some embodiments, the chemotherapeutic agent is ofatumumab. In some embodiments, the individual has received previous anticancer therapy. In some embodiments, the individual has not received previous anticancer therapy.

Disclosed herein, in certain embodiments, is a method of assessing whether an individual having a disease or condition characterized by an increase in the expression level of miR-155 is responsive or likely to be responsive to therapy with a BTK inhibitor (e.g., an irreversible BTK inhibitor such as ibrutinib), comprising: (a) administering a treatment comprising the BTK inhibitor; (b) determining an expression level of miR-155 in a sample from the individual following administration of the treatment; and (c) characterizing the individual as responsive or likely to be responsive to therapy if the individual shows a decrease in the expression level of miR-155 relative to a control. In some embodiments, the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with the BTK inhibitor. In some embodiments, the control is the expression level of miR-155 in the individual prior to treatment with the BTK inhibitor. In some embodiments, the expression level of miR-155 is measured on day 1, 2, 3, 4, 5, 6, 7, 8, 9, 10, 12, 14, 16, 18, 20, 25, 29, or more following treatment with the BTK inhibitor. In some embodiments, the disease or condition characterized by an increase in the expression level of miR-155 is cancer, an inflammatory disorder or an autoimmune disorder. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the methods further comprise detection of the nucleic acid using a microarray. In some embodiments, the methods further comprise amplification of the nucleic acid. In some embodiments, the amplification is a polymerase chain reaction. In some embodiments, the treatment further comprises a second therapy. In some embodiments, the second therapy is a chemotherapeutic agent or an anti-inflammatory agent. In some embodiments, the individual has received previous anticancer therapy. In some embodiments, the individual has not received previous anticancer therapy. In some embodiments, the BTK inhibitor is administered at a dosage of about 40 mg/day to about 1000 mg/day. In some embodiments, the BTK inhibitor is administered orally. In some embodiments, the BTK inhibitor is administered once a day, two times per day, three times per day, four times per day, or five times per day.

Disclosed herein, in certain embodiments, is a method of treating an individual having a disease or condition characterized by an increase in the expression level of miR-155, comprising: (a) administering a treatment comprising a BTK inhibitor (e.g., an irreversible BTK inhibitor such as ibrutinib); (b) determining an expression level of miR-155 in a sample from the individual following administration of the treatment; and (c) continuing the treatment if the expression level of miR-155 is decreased by a predetermined amount relative to the expression level of miR-155 prior to the treatment. Further disclosed herein, in certain embodiments, is a method of treating an individual having a disease or condition characterized by an increase in the expression level of miR-155, comprising: (a) administering a treatment comprising a BTK inhibitor (e.g., an irreversible BTK inhibitor such as ibrutinib); (b) determining an expression level of miR-155 in a sample from the individual following administration of the treatment; and (c) discontinuing the treatment if the expression level of miR-155 is not decreased by a predetermined amount relative to the expression level of miR-155 prior to the treatment. In some embodiments, the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with the BTK inhibitor. Also disclosed herein, in certain embodiments, is a method of optimizing the treatment of a disease or condition characterized by an increase in the expression level of miR-155 in an individual in need thereof, comprising: (a) administering a treatment comprising a BTK inhibitor (e.g., an irreversible BTK inhibitor such as ibrutinib); (b) determining an expression level of miR-155 in a sample from the individual following administration of the treatment; and (c) modifying the treatment based on the expression level of miR-155 relative to a control. In some embodiments, the control is the expression level of miR-155 in the individual prior to treatment with the BTK inhibitor. In some embodiments, the expression level of miR-155 is measured on day 1, 2, 3, 4, 5, 6, 7, 8, 9, 10, 12, 14, 16, 18, 20, 25, 29, or more following treatment with the BTK inhibitor. In some embodiments, the disease or condition characterized by an increase in the expression level of miR-155 is cancer, an inflammatory disorder or an autoimmune disorder. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the methods further comprise detection of the nucleic acid using a microarray. In some embodiments, the methods further comprise amplification of the nucleic acid. In some embodiments, the amplification is a polymerase chain reaction. In some embodiments, the treatment further comprises a second therapy. In some embodiments, the second therapy is a chemotherapeutic agent or an anti-inflammatory agent. In some embodiments, the individual has received previous anticancer therapy. In some embodiments, the individual has not received previous anticancer therapy. In some embodiments, the BTK inhibitor is administered at a dosage of about 40 mg/day to about 1000 mg/day. In some embodiments, the BTK inhibitor is administered orally. In some embodiments, the BTK inhibitor is administered once a day, two times per day, three times per day, four times per day, or five times per day.

Disclosed herein, in certain embodiments, is a method of selecting an individual having a disease or condition characterized by an increase in the expression level of miR-155 for therapy with a BTK inhibitor (e.g., an irreversible BTK inhibitor such as ibrutinib), comprising: (a) measuring the expression level of miR-155 in a sample from the individual; (b) comparing the expression level of miR-155 with a reference level; and (c) characterizing the individual as a candidate for therapy with the BTK inhibitor if the individual has an elevated level of miR-155 compared to the reference level. In some embodiments, the elevated level of miR-155 is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher in the expression of miR-155. In some embodiments, the reference level is the expression level of miR-155 in an individual who does not have the disease or condition. In some embodiments, the disease or condition characterized by an increase in the expression level of miR-155 is cancer, an inflammatory disorder or an autoimmune disorder. In some embodiments, the sample is a blood sample or a serum sample. In some embodiments, determining the expression level of miR-155 in the sample comprises measuring the amount of nucleic acid encoding miR-155 in the sample. In some embodiments, the sample comprises one or more tumor cells. In some embodiments, the nucleic acid is mRNA. In some embodiments, the methods further comprise detection of the nucleic acid using a microarray. In some embodiments, the methods further comprise amplification of the nucleic acid. In some embodiments, the amplification is a polymerase chain reaction. In some embodiments, the treatment further comprises a second therapy. In some embodiments, the second therapy is a chemotherapeutic agent or an anti-inflammatory agent. In some embodiments, the individual has received previous anticancer therapy. In some embodiments, the individual has not received previous anticancer therapy.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates Kaplan-Meier curves of progression-free survival according to low and high levels of miR-155 expression in relapse/refractory CLL patients prior to treatment with chemoimmunotherapy. FIG. 1B illustrates Kaplan-Meier curves of overall survival according to low and high levels of miR-155 expression in relapse/refractory CLL patients prior to treatment with chemoimmunotherapy.

FIG. 2A illustrates miR-155 expression at pre-treatment, 8 days (C1D8), and 29 days (C2D1) of treatment with ibrutinib. FIG. 2B illustrates miR-155 expression at pre-treatment and 29 days (C2D1) of treatment with ibrutinib; miR-155 expression was significantly down-regulated at C2D1 (p=0.0006) relative to pre-treatment. n=34. FIG. 2C illustrates miR-155 expression at pre-treatment, 29 days (C2D1) and 1 year (C12D1) of therapy in 5 patients with partial response with persistent blood lymphocytosis; miR-155 expression was significantly decreased at C2D1 (p=0.005) and at C12D1 (p=0.013) relative to pre-treatment. FIG. 2D illustrates miR-155 expression at pre-treatment, time of response, and time of relapse in 4 patients treated with ibrutinib; miR-155 expression was significantly decreased at time of response (p=0.002) but significantly increased at relapse (p=0.002) relative to pre-treatment.

### DETAILED DESCRIPTION OF THE INVENTION

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose.

As used herein, the term “refractory” refers to an abolishment of a response or a development of an acquired resistance to a disease in a subject to a particular course of treatment.

As used herein, the term “treatment” refers to stopping the progression of a disease, partial or complete elimination of a disease, reversing progression of a disease, stopping, reducing or reversing episodes of worsening or relapses of a disease, or prolonging episodes of remission of a disease in a subject.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g., constant or intermittent) of a health care worker (e.g., a doctor, a registered nurse, a nurse practitioner, a physician's assistant, an orderly or a hospice worker).

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that, in some instances, are present in minor amounts.

Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these are further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

As used herein, a control refers to the expression level of miR-155 in a sample that is substantially identical to the test sample, except that it is not treated with a test parameter, or, is prior to the treatment of the test parameter. In some embodiments, a control is an internal control. In some embodiments, a control is from a recombinant cell line. In some embodiments, a control is from a CLL cell line. In some embodiments, a control is from a normal patient not affected with the condition of interest. In some embodiments, this control is also referred to as a reference level. In some embodiments, the reference level is the expression level of miR-155 in a sample from a normal patient not affected with the condition of interest.

As used herein, the term “biomarker(s)” is a generic term referring to any biological molecules found either in blood, other body fluids, or tissues. A non-exhaustive list of biomarkers and markers include: ZAP70, t(14,18), 13-2 microglobulin, p53 mutational status, ATM mutational status, del(17)p, del(11)q, del(6)q, CD3, CD4, CD5, CD11c, CD19, CD20, CD22, CD25, CD26, CD28, CD30, CD33, CD38, CD45, CD52, CD62, CD81, CD94, CD103, CD119, CD152, CD138, CD183, CD184, CD191 (CCR1), CD195, CD197 (CCR7), CD212, CD278, CCR3, CCR4, CCR8, TBX21, NKG7, XCL1 (lymphotactin), TXK, GZMB (granzyme B), S100P, LIR9, KIR3DL2, VAV3, DLG5, MMP-9, MS4A4A, lymphotoxin, perforin, t-bet, Tim-1, Tim-3, TRANCE, GATA-3, c-maf, CRTH2, ST2L/T1, secreted, surface or cytoplasmic immunoglobulin expression, V_{H} mutation status; chemokines such as GCP-2 (granulocyte chemotactic protein 2), Gro-a (growth related oncogene a), Gro-β (growth related oncogene β), Gro-γ (growth related oncogene γ), NAP-2 (neutrophil activating protein), (epithelial-cell-derived neutrophil-activating chemokine), IP-10 (Interferon-inducible protein-10), (monokine induced by interferone γ), 1-TAC (Interferon-inducible T-cell alpha chemoattractant), SDF-1 (stromal cell-derived factor-1), PBSF (pre-B-cell growth stimulating factor), BCA-1 (B-lymphocyte chemoattractant 1), MIP-1 (macrophage inflammatory protein 1), RANTES (regulated upon activation, normal T-cell expressed and secreted), MIP-5 (macrophage inflammatory protein 5), MCP-1 (monocyte chemoattractant protein 1), MCP-2 (monocyte chemoattractant protein 2), MCP-3 (monocyte chemoattractant protein 3), MCP-4 (monocyte chemoattractant protein 4), Eotaxin, TARC (thymus- and acticvation-regulated chemokine), MIP-1 a (macrophage inflammatory protein 1a), MIP-1β (macrophage inflammatory protein 1β), Exodus-1, ELC (Eb11 ligand chemokine); cytokines such as lymphokines, monokines, traditional polypeptide hormones, growth hormone (e.g., human growth hormone, N-methionyl human growth hormone, bovine growth hormone); parathyroid hormone; thyroxine; insulin; proinsulin; relaxin; prorelaxin; glycoprotein hormones (e.g., follicle stimulating hormone (FSH), thyroid stimulating hormone (TSH) and luteinizing hormone (LH)); epidermal growth factor; hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) (e.g., TGF-alpha and TGF-beta); insulin-like growth factor-I and -II; erythropoietin (EPO); osteoinductive factors; interferons (e.g., interferon-alpha, -beta and -gamma); colony stimulating factors (CSFs) (e.g., macrophage-CSF (M-CSF), granulocyte-macrophage-CSF (GM-CSF) and granulocyte-CSF (G-CSF)); interleukins (ILs) (e.g., IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL 20, IL-21, IL-22, IL-23, IL 24, IL-25, IL-26, IL 27, IL-28, IL, 29, IL-32, IL-33, IL-35 and IL-36); a tumor necrosis factor (e.g., TNF-alpha and TNF-beta) and other polypeptide factors including LIF and kit ligand (KL). As used herein, the terms biomarker and marker include proteins from natural sources or from recombinant cell culture and biologically active equivalents of the native sequence biomarkers/markers.

As used herein, the term “cancer” refers to or describe the physiological condition in mammals that is typically characterized by unregulated cell growth. Included in this definition are benign and malignant cancers as well as dormant tumors or micrometastatses. The term cancer includes solid tumors and hematologic cancers. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, ovarian cancer, thyroid cancer, proximal or distal bile duct carcinoma, hepatic carcinoma and various types of head and neck cancer, T-cell lymphoma, as well as B-cell lymphoma, including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

### MicroRNAs and Cancer

MicroRNAs (miRNAs) are non-coding RNAs that control gene expression either by degradation of target mRNAs or by post-transcriptional repression. MicroRNA (miR) expression profiling in hematological malignancies and solid tumors have identified several miRs that are associated with prognosis and pathogenesis. For example, miR profiling in CLL has identified several miRs that are associated with shorter time to treatment from diagnosis, such as high expression of miR-155 and miR-181a and low expression of miR-29c. Additionally, in fludarabine-treated CLL patients, pre-treatment expression of miR-148a, miR-21 and miR-222 are associated with clinical response to fludarabine. In addition, in a profiling study on solid tumors including lung, breast, stomach, prostate, colon, and pancreatic tumors, miR profiling shows overexpressions of miR-17-5p, miR-20a, miR-21, miR-92, miR-106a and miR-155, which have been attributed to be involved in cancer pathogenesis and support their functions by modulating the expression of protein-coding tumor suppressors and oncogenes.

MiR-155 regulates hematopoietic cell development and along with its host gene BIC, is indicated to be overexpressed in hematological malignancies and solid tumors. In a mouse study, miR-155 has been found to be leukemogeneic when overexpressed under a B cell specific promoter. Notably, in normal B-cells, miR-155 has been shown to increase following B-cell receptor (BCR) activation. Further, the ABC subtype of diffused large B cell lymphoma (DLBCL), which the patients have a poor prognosis compared to other subtypes of DLBCL, has a 2 to 3 fold higher expression level of miR-155 than the GC-DLBCL subtype.

### Chronic Lymphocytic Leukemia (CLL)

Chronic lymphoid leukemia (CLL), or B-cell CLL, is the most common hematological malignancy in adults. It is estimated that 100,760 people in the United States are living with or are in remission from CLL. Most (>75%) people newly diagnosed with CLL are over the age of 50. CLL is characterized by a heterogeneous clinical course, exemplified with either indolent disease or aggressive clinical outcome. Poor prognosis is generally associated with negative prognostic factors such as the expression and methylation of ZAP70 or CD38, the presence of chromosome abnormalities including 17p and/or 11q, the absence of somatic mutations in the immunoglobulin heavy chain variable (IGHV) gene, and the up-regulation/down-regulation of non-coding microRNAs (miRNAs) including miR-155.

In CLL, the expression level of miR-155 is up-regulated. Further, in MEC1 cell line studies using a miR antagomiR or locked nucleic acid complementary to miR-155, it has been demonstrated that neutralizing miR-155 function lead to inhibition of proliferation, but not induction of apoptosis. In addition, the overexpression of miR-155 in CLL has been correlated to an absence of somatic mutations in IGHV and low ZAP70 methylation. Therefore, in certain embodiments provided herein, the expression level of miR-155 is used as a prognostic factor or biomarker for CLL. Further, in certain embodiments provided herein, the expression level of miR-155 in CLL is used as a biomarker for assessing, optimizing, or modifying treatment with ibrutinib.

Ibrutinib (PCI-32765) is an irreversible covalent inhibitor of Bruton's tyrosine kinase (Btk), a key signaling enzyme in the BCR pathway. Ibrutinib has been shown to inhibit proliferation, induce apoptosis, and has been shown to inhibit Btk in animal models. In in vitro analysis of primary CLL cells, ibrutinib has been shown to decrease pro-survival signaling, such as AKT, ERK and NFκB. Further, clinical trials have demonstrated efficacy in CLL. Indeed, about 70% of CLL patient have demonstrated an objective complete or partial response in a clinical trial and an additional 15 to 20% of patients have a partial response with persistent lymphocytosis.

Disclosed herein, in certain embodiments, is a method of assessing whether an individual having chronic lymphocytic leukemia (CLL) is responsive or likely to be responsive to therapy with ibrutinib, comprising: (a) administering a treatment comprising ibrutinib; (b) determining an expression level of miR-155 in a sample from the individual following administration of the treatment; and (c) characterizing the individual as responsive or likely to be responsive to therapy if the individual shows a decrease in the expression level of miR-155 relative to a control. Further disclosed herein, in certain embodiments, is a method of monitoring whether an individual receiving ibrutinib for treatment of chronic lymphocytic leukemia (CLL) has relapsed or is likely to have a relapse to therapy, comprising: (a) administering a treatment comprising ibrutinib; (b) determining an expression level of miR-155 in a sample from the individual following administration of the treatment; and (c) characterizing the individual as relapsed or likely to have a relapse to therapy if the individual does not show a decrease in the expression level of miR-155 relative to a control.

Disclosed herein, in certain embodiments, is a method of treating an individual having chronic lymphocytic leukemia (CLL), comprising: (a) administering a treatment comprising ibrutinib; (b) determining an expression level of miR-155 in a sample from the individual following administration of the treatment; and (c) continuing the treatment if the expression level of miR-155 is decreased by a predetermined amount relative to the expression level of miR-155 prior to the treatment. Further disclosed herein, in certain embodiments, is a method of treating an individual having chronic lymphocytic leukemia (CLL), comprising: (a) administering a treatment comprising ibrutinib; (b) determining an expression level of miR-155 in a sample from the individual following administration of the treatment; and (c) discontinuing the treatment if the expression level of miR-155 is not decreased by a predetermined amount relative to the expression level of miR-155 prior to the treatment. Also disclosed herein, in certain embodiments, is a method of optimizing the treatment of chronic lymphocytic leukemia (CLL) in an individual in need thereof, comprising: (a) administering a treatment comprising ibrutinib; (b) determining an expression level of miR-155 in a sample from the individual following administration of the treatment; and (c) modifying the treatment based on the expression level of miR-155 relative to a control.

Disclosed herein, in certain embodiments, is a method of selecting an individual having chronic lymphocytic leukemia (CLL) for therapy with ibrutinib, comprising: (a) measuring the expression level of miR-155 in a sample from the individual; (b) comparing the expression level of miR-155 with a reference level; and (c) characterizing the individual as a candidate for therapy with ibrutinib if the individual has an elevated level of miR-155 compared to the reference level.

### CLL Classifications by Staging, Cytogenetic Abnormalities and Associated Diseases

In some embodiments, CLL is classified by staging. In some embodiments, the staging utilizes a Binet system. In some embodiments, the staging utilizes a Rai system. In some embodiments, the Rai staging is further categorized into five stages. In some embodiments, the Rai stages comprise Rai stage 0, Rai stage I, Rai stage II, Rai stage III, and Rai stage IV. In some embodiments, Rai stage 0 is characterized by lymphocytosis without enlargement of the lymph nodes, spleen, or liver, and with near normal red blood cell and platelet counts. In some embodiments, Rai stage I is characterized by lymphocytosis with enlarged lymph nodes. In some embodiments, Rai stage I is further characterized with normal sized spleen and liver and near normal red blood cell and platelet counts. In some embodiments, Rai stage II is characterized by lymphocytosis, enlarged spleen, and potentially enlarged liver and enlarged lymph nodes. In some embodiments, the red blood cell and platelet counts are near normal. In some embodiments, Rai stage III is characterized by lymphocytosis, anemia, and potentially enlarged lymph nodes, spleen, or liver. In some embodiments, the platelet counts are near normal. In some embodiments, Rai stage IV is characterized by lymphocytosis and thrombocytopenia, potentially anemia, and enlarged lymph nodes, spleen, or liver. In some embodiments, Rai stage 0 is classified as low risk. In some embodiments, Rai stages I and II are classified as intermediate risk. In some embodiments, Rai stages III and IV are classified as high risk.

In some embodiments, CLL is characterized by cytogenetic abnormalities. In some embodiments, the cytogenetic abnormalities include del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, or a combination thereof. In some embodiments, the cytogenetic abnormality is del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, or a combination thereof. As used herein, “complex karyotype” means the abnormalities of three or more chromosomes excluding chromosome 17. In some embodiments, CLL is also classified as high-risk. In some embodiments, high-risk CLL is characterized by one or more cytogenetic abnormalities including del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, or a combination thereof.

In some embodiments, the expression level of miR-155 is associated with the presence or the level of one or more cytogenetic abnormalities. In some embodiments, the expression level of miR-155 is associated with one or more cytogenetic abnormalities selected from del(17p13.1), del(11q22.3), del(11q23), unmutated IgVH together with ZAP-70+ and/or CD38+, trisomy 12, del(13q14), +(12q21), del(6q21), ATM del, p53 del, and complex karyotype. In some embodiments, the expression level of miR-155 is associated with unmutated IgVH and ZAP-70 methylation. In some embodiments, the expression level of miR-155 is associated with unmutated IgVH. In some embodiments, the expression level of miR-155 is associated with ZAP-70 methylation. In some embodiments, the expression level of miR-155 is associated with a low ZAP-70 methylation.

In some embodiments, the expression level of miR-155 is a “high expression level”. In some embodiments, the “high expression level” of miR-155 in an individual refers to an elevated level of miR-155 relative to normal expression. In some embodiments, the “high expression level” of miR-155 is a 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher in the expression of miR-155 in the individual relative to normal expression.

In some embodiments, the expression level of miR-155 is a “low expression level”. In some embodiments, the “low expression level” of miR-155 in an individual refers to a level of miR-155 relative to normal expression. In some embodiments, the level is an elevated level of miR-155 relative to normal expression. In some embodiments, the ‘low expression level” of miR-155 is less than 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold, in the expression of miR-155 in the individual relative to normal expression.

In some embodiments, the “high expression level” of miR-155 is associated with the presence or level of one or more cytogenetic abnormalities. In some embodiments, the “high expression level” of miR-155 is associated with one or more cytogenetic abnormalities selected from del(17p13.1), del(11q22.3), del(11q23), unmutated IgVH together with ZAP-70+ and/or CD38+, trisomy 12, del(13q14), +(12q21), del(6q21), ATM del, p53 del, and complex karyotype. In some embodiments, the “high expression level” of miR-155 is associated with unmutated IgVH and ZAP-70 methylation. In some embodiments, the “high expression level” of miR-155 is associated with unmutated IgVH. In some embodiments, the “high expression level” of miR-155 is associated with ZAP-70 methylation. In some embodiments, the “high expression level” of miR-155 is associated with a low ZAP-70 methylation.

In some embodiments, the expression level of miR-155 is independent of the presence of cytogenetic abnormalities or Rai stages. In some embodiments, the expression level of miR-155 is independent of the presence of cytogenetic abnormalities such as del(17p) and/or del(11p). In some embodiments, the expression level of miR-155 is independent of Rai stages.

In some embodiments, the expression level of miR-155 correlates to progression free survival (PFS) and overall survival (OS). In some embodiments, the “high expression level” of miR-155 correlates to PFS and OS. In some embodiments, the “high expression level” of miR-155 correlates to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, or more months for PFS. In some embodiments, the “high expression level” of miR-155 correlates to about less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60 months for PFS. In some embodiments, the “high expression level” of miR-155 correlates to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more months for OS. In some embodiments, the “high expression level” of miR-155 correlates to about less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 months for OS.

In some embodiments, the “low expression level” of miR-155 correlates to PFS and OS. In some embodiments, the “low expression level” of miR-155 correlates to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, or more months for PFS. In some embodiments, the “low expression level” of miR-155 correlates to about less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, or 70 months for PFS. In some embodiments, the “low expression level” of miR-155 correlates to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more months for OS. In some embodiments, the “low expression level” of miR-155 correlates to about less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 months for OS.

CLL and small lymphocytic lymphoma (SLL) are commonly thought as the same disease with different manifestations, and are determined based on the location of the cancerous cells. When the cancer cells are primarily found in the lymph nodes, lima bean shaped structures of the lymphatic system (a system primarily of tiny vessels found in the body), it is called SLL. SLL accounts for about 5% to 10% of all lymphomas. When the cancer cells are primarily found in the bloodstream and the bone marrow, it is called CLL. In some embodiments, the expression level of miR-155 is used as a prognostic factor for SLL. In some embodiments, the “high expression level” of miR-155 is used as a prognostic factor for SLL. In some embodiments, the expression level of miR-155 is used as a prognostic factor for modulating an ibrutinib-based therapy or optimizing an ibrutinib-based therapy for an individual having SLL. In some embodiments, the expression level of miR-155 is used to assess whether an individual having SLL is responsive or likely to be responsive to therapy with ibrutinib. In some embodiments, the expression level of miR-155 is used to monitor whether an individual receiving ibrutinib for treatment of SLL has relapsed or is likely to have a relapse to therapy. In some embodiments, the expression level of miR-155 is used as a prognostic factor in selecting an individual having SLL for ibrutinib-based therapy.

Richter's transformation or Richter's syndrome (RS) is a complication of CLL in which the leukemia changes into a fast-growing diffuse large B cell lymphoma. In general, about 5% of the CLL patients are affected by Richter's transformation. In some embodiments, the expression level of miR-155 is used as a prognostic factor for Richter's transformation. In some embodiments, the “high expression level” of miR-155 is used as a prognostic factor for Richter's transformation. In some embodiments, the expression level of miR-155 is used as a prognostic factor for modulating an ibrutinib-based therapy or optimizing an ibrutinib-based therapy for an individual having Richter's transformation. In some embodiments, the expression level of miR-155 is used to assess whether an individual having Richter's transformation is responsive or likely to be responsive to therapy with ibrutinib. In some embodiments, the expression level of miR-155 is used to monitor whether an individual receiving ibrutinib for treatment of Richter's transformation has relapsed or is likely to have a relapse to therapy. In some embodiments, the expression level of miR-155 is used as a prognostic factor in selecting an individual having Richter's transformation for ibrutinib-based therapy.

In some embodiments, CLL is a relapsed or refractory CLL. In some embodiments, CLL is a relapsed CLL. In some embodiments, CLL is a refractory CLL. In some embodiments, the expression level of miR-155 is used as a prognostic factor for relapsed or refractory CLL. In some embodiments, the “high expression level” of miR-155 is used as a prognostic factor for relapsed or refractory CLL. In some embodiments, the expression level of miR-155 is used as a prognostic factor for modulating an ibrutinib-based therapy or optimizing an ibrutinib-based therapy for an individual having relapsed or refractory CLL. In some embodiments, the expression level of miR-155 is used to assess whether an individual having relapsed or refractory CLL is responsive or likely to be responsive to therapy with ibrutinib. In some embodiments, the expression level of miR-155 is used to monitor whether an individual receiving ibrutinib for treatment of relapsed or refractory CLL has relapsed or is likely to have a relapse to therapy. In some embodiments, the expression level of miR-155 is used as a prognostic factor in selecting an individual having relapsed or refractory CLL for ibrutinib-based therapy.

### Additional Cancers

Solid tumor refers to an abnormal mass or tissue as a result of abnormal growth or division of cells. In some embodiments, a solid tumor is a sarcoma or carcinoma. In some embodiments, the solid tumor is a sarcoma. In some embodiments, the sarcoma is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. In some embodiments, the carcinoma is selected from an adenocarcinoma, squamous cell carcinoma, adenosquamous carcinoma, anaplastic carcinoma, large cell carcinoma, or small cell carcinoma. In some embodiments, the carcinoma is selected from anal cancer; appendix cancer; bile duct cancer (i.e., cholangiocarcinoma); bladder cancer; brain tumor; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer.

Hematological malignancy is a diverse group of cancer that affects the blood, bone marrow, and lymph nodes. In some embodiments, the hematologic malignancy is a leukemia, a lymphoma, a myeloma, a non-Hodgkin's lymphoma, a Hodgkin's lymphoma, or a B-cell malignancy. In some embodiments, hematological malignancy is chronic lymphocytic leukemia (CLL), small lymphocytic lymphoma (SLL), high risk CLL, or a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL) and germinal center diffuse large B-cell lymphoma (GCB DLBCL). In some embodiments, the hematological malignancy is a relapsed or refractory hematological malignancy.

Disclosed herein, in certain embodiments, are methods and diagnosis of treating an individual having a solid tumor with a BTK inhibitor and modify or optimize the treatment with a BTK inhibitor based on the expression level of miR-155. In some embodiments, disclosed herein are methods of assessing whether an individual having a solid tumor is responsive or likely to be responsive to therapy with a BTK inhibitor based on the expression level of miR-155. In some embodiments, disclosed herein are methods of assessing or monitoring the efficacy of the treatment with a BTK inhibitor in an individual having a solid tumor based on the expression level of miR-155. In some embodiments, disclosed herein are methods of selecting patients having a solid tumor as candidates for ibrutinib therapy based on the expression of miR-155. In some embodiments, the expression level of miR-155 and at least one additional biomarkers are determined. In some embodiments, the solid tumor is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. adenocarcinoma, squamous cell carcinoma, adenosquamous carcinoma, anaplastic carcinoma, large cell carcinoma, or small cell carcinoma; anal cancer; appendix cancer; bile duct cancer (i.e., cholangiocarcinoma); bladder cancer; brain tumor; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer. In some embodiments, the BTK inhibitor is ibrutinib

Disclosed herein, in certain embodiments, are methods and diagnosis of treating an individual having a solid tumor with ibrutinib and modify or optimize ibrutinib treatment based on the expression level of miR-155. In some embodiments, disclosed herein are methods of assessing whether an individual having a solid tumor is responsive or likely to be responsive to therapy with ibrutinib based on the expression level of miR-155. In some embodiments, disclosed herein are methods of assessing or monitoring the efficacy of the ibrutinib treatment in an individual having a solid tumor based on the expression level of miR-155. In some embodiments, disclosed herein are methods of selecting patients having a solid tumor as candidates for ibrutinib therapy based on the expression of miR-155. In some embodiments, the expression level of miR-155 and at least one additional biomarkers are determined. In some embodiments, the solid tumor is selected from alveolar rhabdomyosarcoma; alveolar soft part sarcoma; ameloblastoma; angiosarcoma; chondrosarcoma; chordoma; clear cell sarcoma of soft tissue; dedifferentiated liposarcoma; desmoid; desmoplastic small round cell tumor; embryonal rhabdomyosarcoma; epithelioid fibrosarcoma; epithelioid hemangioendothelioma; epithelioid sarcoma; esthesioneuroblastoma; Ewing sarcoma; extrarenal rhabdoid tumor; extraskeletal myxoid chondrosarcoma; extraskeletal osteosarcoma; fibrosarcoma; giant cell tumor; hemangiopericytoma; infantile fibrosarcoma; inflammatory myofibroblastic tumor; Kaposi sarcoma; leiomyosarcoma of bone; liposarcoma; liposarcoma of bone; malignant fibrous histiocytoma (MFH); malignant fibrous histiocytoma (MFH) of bone; malignant mesenchymoma; malignant peripheral nerve sheath tumor; mesenchymal chondrosarcoma; myxofibrosarcoma; myxoid liposarcoma; myxoinflammatory fibroblastic sarcoma; neoplasms with perivascular epitheioid cell differentiation; osteosarcoma; parosteal osteosarcoma; neoplasm with perivascular epitheioid cell differentiation; periosteal osteosarcoma; pleomorphic liposarcoma; pleomorphic rhabdomyosarcoma; PNET/extraskeletal Ewing tumor; rhabdomyosarcoma; round cell liposarcoma; small cell osteosarcoma; solitary fibrous tumor; synovial sarcoma; telangiectatic osteosarcoma. adenocarcinoma, squamous cell carcinoma, adenosquamous carcinoma, anaplastic carcinoma, large cell carcinoma, or small cell carcinoma; anal cancer; appendix cancer; bile duct cancer (i.e., cholangiocarcinoma); bladder cancer; brain tumor; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; or vulvar cancer.

Disclosed herein, in certain embodiments, are methods and diagnosis of treating an individual having a hematological malignancy with a BTK inhibitor and modify or optimize the treatment with a BTK inhibitor based on the expression level of miR-155. In some embodiments, disclosed herein are methods of assessing whether an individual having a hematological malignancy is responsive or likely to be responsive to therapy with a BTK inhibitor based on the expression level of miR-155. In some embodiments, disclosed herein are methods of assessing or monitoring the efficacy of the treatment with a BTK inhibitor in an individual having a hematological malignancy based on the expression level of miR-155. In some embodiments, disclosed herein are methods of selecting patients having a hematological malignancy as candidates for therapy with a BTK inhibitor based on the expression of miR-155. In some embodiments, the expression level of miR-155 and at least one additional biomarkers are determined. In some embodiments, the hematologic malignancy is a leukemia, a lymphoma, a myeloma, a non-Hodgkin's lymphoma, a Hodgkin's lymphoma, or a B-cell malignancy. In some embodiments, hematological malignancy is chronic lymphocytic leukemia (CLL), small lymphocytic lymphoma (SLL), high risk CLL, or a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL) and germinal center diffuse large B-cell lymphoma (GCB DLBCL). In some embodiments, the hematological malignancy is a relapsed or refractory hematological malignancy. In some embodiments, the BTK inhibitor is ibrutinib.

Disclosed herein, in certain embodiments, are methods and diagnosis of treating an individual having a hematological malignancy with ibrutinib and modify or optimize ibrutinib treatment based on the expression level of miR-155. In some embodiments, disclosed herein are methods of assessing whether an individual having a hematological malignancy is responsive or likely to be responsive to therapy with ibrutinib based on the expression level of miR-155. In some embodiments, disclosed herein are methods of assessing or monitoring the efficacy of the ibrutinib treatment in an individual having a hematological malignancy based on the expression level of miR-155. In some embodiments, disclosed herein are methods of selecting patients having a hematological malignancy as candidates for ibrutinib therapy based on the expression of miR-155. In some embodiments, the expression level of miR-155 and at least one additional biomarkers are determined. In some embodiments, the hematologic malignancy is a leukemia, a lymphoma, a myeloma, a non-Hodgkin's lymphoma, a Hodgkin's lymphoma, or a B-cell malignancy. In some embodiments, hematological malignancy is chronic lymphocytic leukemia (CLL), small lymphocytic lymphoma (SLL), high risk CLL, or a non-CLL/SLL lymphoma. In some embodiments, the cancer is follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, DLBCL is further divided into subtypes: activated B-cell diffuse large B-cell lymphoma (ABC-DLBCL) and germinal center diffuse large B-cell lymphoma (GCB DLBCL). In some embodiments, the hematological malignancy is a relapsed or refractory hematological malignancy.

### Diagnostic and Therapeutic Methods

Disclosed herein, in certain embodiments, is a method of assessing whether an individual having chronic lymphocytic leukemia (CLL) is responsive or likely to be responsive to therapy with ibrutinib, comprising: (a) administering a treatment comprising ibrutinib; (b) determining an expression level of miR-155 in a sample from the individual following administration of the treatment; and (c) characterizing the individual as responsive or likely to be responsive to therapy if the individual shows a decrease in the expression level of miR-155 relative to a control. Further disclosed herein, in certain embodiments, is a method of monitoring whether an individual receiving ibrutinib for treatment of chronic lymphocytic leukemia (CLL) has relapsed or is likely to have a relapse to therapy, comprising: (a) administering a treatment comprising ibrutinib; (b) determining an expression level of miR-155 in a sample from the individual following administration of the treatment; and (c) characterizing the individual as relapsed or likely to have a relapse to therapy if the individual does not show a decrease in the expression level of miR-155 relative to a control. In some embodiments, the expression level of miR-155 decreases by about 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the control is the expression level of miR-155 in the individual prior to treatment with ibrutinib. In some embodiments, the expression level of miR-155 is measured on day 1, 2, 3, 4, 5, 6, 7, 8, 9, 10, 12, 14, 16, 18, 20, 25, 29, or more following treatment with ibrutinib. In some embodiments, the individual has received previous anticancer therapy prior to treatment with ibrutinib. In some embodiments, the individual has not received previous anticancer therapy prior to treatment with ibrutinib.

Disclosed herein, in certain embodiments, is a method of treating an individual having chronic lymphocytic leukemia (CLL), comprising: (a) administering a treatment comprising ibrutinib; (b) determining an expression level of miR-155 in a sample from the individual following administration of the treatment; and (c) continuing the treatment if the expression level of miR-155 is decreased by a predetermined amount relative to the expression level of miR-155 prior to the treatment. Further disclosed herein, in certain embodiments, is a method of treating an individual having chronic lymphocytic leukemia (CLL), comprising: (a) administering a treatment comprising ibrutinib; (b) determining an expression level of miR-155 in a sample from the individual following administration of the treatment; and (c) discontinuing the treatment if the expression level of miR-155 is not decreased by a predetermined amount relative to the expression level of miR-155 prior to the treatment. Also disclosed herein, in certain embodiments, is a method of optimizing the treatment of chronic lymphocytic leukemia (CLL) in an individual in need thereof, comprising: (a) administering a treatment comprising ibrutinib; (b) determining an expression level of miR-155 in a sample from the individual following administration of the treatment; and (c) modifying the treatment based on the expression level of miR-155 relative to a control. In some embodiments, the expression level of miR-155 decreases by about 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib. In some embodiments, the control is the expression level of miR-155 in the individual prior to treatment with ibrutinib. In some embodiments, the expression level of miR-155 is measured on day 1, 2, 3, 4, 5, 6, 7, 8, 9, 10, 12, 14, 16, 18, 20, 25, 29, or more following treatment with ibrutinib. In some embodiments, the individual has received previous anticancer therapy prior to treatment with ibrutinib. In some embodiments, the individual has not received previous anticancer therapy prior to treatment with ibrutinib.

Disclosed herein, in certain embodiments, is a method of selecting an individual having chronic lymphocytic leukemia (CLL) for therapy with ibrutinib, comprising: (a) measuring the expression level of miR-155 in a sample from the individual; (b) comparing the expression level of miR-155 with a reference level; and (c) characterizing the individual as a candidate for therapy with ibrutinib if the individual has an elevated level of miR-155 compared to the reference level. In some embodiments, the elevated level of miR-155 is about 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher in the expression of miR-155. In some embodiments, the elevated level of miR-155 is 1-fold, 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or higher in the expression of miR-155. In some embodiments, the reference level is the expression level of miR-155 in an individual who does not have CLL. In some embodiments, the expression level of miR-155 is measured on day 1, 2, 3, 4, 5, 6, 7, 8, 9, 10, 12, 14, 16, 18, 20, 25, 29, or more following treatment with ibrutinib. In some embodiments, the individual has received previous anticancer therapy prior to treatment with ibrutinib. In some embodiments, the individual has not received previous anticancer therapy prior to treatment with ibrutinib.

In some embodiments, the treatment with ibrutinib further comprises a second anticancer therapy. Exemplary anticancer agents include but are not limited to, adriamycin (doxorubicin), bexxar, bendamustine, bleomycin, blenoxane, bortezomib, dacarbazine, deltasone, cisplatin, cyclophosphamide, cytoxan, DTIC dacarbazine, dasatinib, doxorubicin, etoposide, fludarabine, granisetron, kytril, lenalidomide, matulane, mechlorethamine, mustargen, mustine, natulan, Rituxan (rituximab, anti-CD20 antibody), VCR, neosar, nitrogen mustard, oncovin, ondansetron, orasone, prednisone, procarbazine, thalidomide, VP-16, velban, velbe, velsar, VePesid, vinblastine, vincristine, Zevalin®, zofran, stem cell transplantation, radiation therapy or combination therapies, such as, for example, ABVD (adriamycin, bleomycin, vinblastine and dacarbazine), ChlvPP (chlorambucil, vinblastine, procarbazine and prednisolone), Stanford V (mustine, doxorubicin, vinblastine, vincristine, bleomycin, etoposide and steroids), BEACOPP (bleomycin, etoposide, doxorubicin, cyclophosphamide, vincristine, procarbazine and prednisolone), BEAM (carmustine (BiCNU) etoposide, cytarabine (Ara-C, cytosine arabinoside), and melphalan), CHOP (cyclophosphamide, doxorubicin, vincristine, and prednisone), R-CHOP (rituximab, doxorubicin, cyclophosphamide, vincristine, and prednisone), EPOCH (etoposide, vincristine, doxorubicin, cyclophosphamide, and prednisone), CVP (cyclophosphamide, vincristine, and prednisone), ICE (ifosfamide-carboplatin-etoposide), R-ACVBP (rituximab, doxorubicin, cyclophosphamide, vindesine, bleomycin, and prednisone), DHAP (dexamethasone, high-dose cytarabine, (Ara C), cisplatin), R-DHAP (rituximab, dexamethasone, high-dose cytarabine, (Ara C), cisplatin), ESHAP (etoposide (VP-16), methyl-prednisolone, and high-dose cytarabine (Ara-C), cisplatin), CDE (cyclophosphamide, doxorubicin and etoposide), Velcade® (bortezomib) plus Doxil® (liposomal doxorubicin), Revlimid® (lenalidomide) plus dexamethasone, and bortezomib plus dexamethasone.

In some embodiments, the anticancer agent is a chemotherapeutic agent or radiation therapy. In some embodiments, the anticancer agent is a chemotherapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the chemotherapeutic agent is selected from among ofatumumab, rituximab, fludarabine, or a combination thereof. In some embodiments, the chemotherapeutic agent is rituximab. In some embodiments, the chemotherapeutic agent is fludarabine. In some embodiments, the chemotherapeutic agent is ofatumumab.

In some embodiments, the individual has received previous anticancer therapy prior to treatment with ibrutinib. In some embodiments, the previous anticancer therapy is a chemotherapeutic agent or radiation therapy. In some embodiments, the previous anticancer agent is a chemotherapeutic agent. In some embodiments, the chemotherapeutic agent is selected from among chlorambucil, ifosfamide, doxorubicin, mesalazine, thalidomide, lenalidomide, temsirolimus, everolimus, fludarabine, fostamatinib, paclitaxel, docetaxel, ofatumumab, rituximab, dexamethasone, prednisone, CAL-101, ibritumomab, tositumomab, bortezomib, pentostatin, endostatin, or a combination thereof. In some embodiments, the chemotherapeutic agent is selected from among ofatumumab, rituximab, fludarabine, or a combination thereof. In some embodiments, the chemotherapeutic agent is rituximab. In some embodiments, the chemotherapeutic agent is fludarabine. In some embodiments, the chemotherapeutic agent is ofatumumab.

In some embodiments, the sample for use in the methods is from any tissue or fluid from a patient. Samples include, but are not limited, to whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In particular embodiments, the sample is a blood serum sample. In particular embodiments, the sample is a tumor biopsy sample. In particular embodiments, the sample is from a fluid or tissue that is part of, or associated with, the lymphatic system or circulatory system. In some embodiments, the sample is a blood sample that is a venous, arterial, peripheral, tissue, cord blood sample. In particular embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g., in a bone marrow aspirate sample).

In some embodiments, the samples are obtained from the individual by any suitable means of obtaining the sample using well-known and routine clinical methods. Procedures for obtaining fluid samples from an individual are well known. For example, procedures for drawing and processing whole blood and lymph are well-known and can be employed to obtain a sample for use in the methods provided. Typically, for collection of a blood sample, an anti-coagulation agent (e.g., EDTA, or citrate and heparin or CPD (citrate, phosphate, dextrose) or comparable substances) is added to the sample to prevent coagulation of the blood. In some examples, the blood sample is collected in a collection tube that contains an amount of EDTA to prevent coagulation of the blood sample.

In some embodiments, the collection of a sample from the individual is performed at regular intervals, such as, for example, one day, two days, three days, four days, five days, six days, one week, two weeks, weeks, four weeks, one month, two months, three months, four months, five months, six months, one year, daily, weekly, bimonthly, quarterly, biyearly or yearly.

In some embodiments, the collection of a sample is performed at a predetermined time or at regular intervals relative to treatment with a BTK inhibitor. For example, a sample is collected from a patient at a predetermined time or at regular intervals prior to, during, or following treatment or between successive treatments with the BTK inhibitor. In particular examples, a sample is obtained from a patient prior to administration of a BTK inhibitor and then again at regular intervals after treatment with the BTK inhibitor has been effected. In some embodiments, the patient is administered a BTK inhibitor and one or more additional anti-cancer agents. In some embodiments, the BTK inhibitor is an irreversible BTK inhibitor. In some embodiments, the BTK inhibitor is a reversible BTK inhibitor. In some embodiments, the BTK inhibitor is ibrutinib. In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK417891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13.

In some embodiments, the individual is administered a BTK inhibitor and one or more additional anticancer agents. In some embodiments, the individual is administered a BTK inhibitor and one or more additional anticancer agents that are not BTK inhibitors. In some embodiments, the patient is administered a BTK inhibitor and one or more additional anticancer agents that are BTK inhibitors. In some embodiments, the individual is administered ibrutinib and one or more additional anticancer agents that are BTK inhibitors. In some embodiments, the individual is administered ibrutinib and one or more additional anticancer agents that are not BTK inhibitors. In some embodiments, the one or more additional anticancer agents include a reversible BTK inhibitor. In some embodiments, the one or more additional anticancer agents include an irreversible BTK inhibitor. In some embodiments, the individual is administered one or more irreversible BTK inhibitors. In some embodiments, the individual is administered one or more reversible BTK inhibitors.

In some embodiments, the individual is administered ibrutinib in combination with one or more reversible BTK inhibitors. For example, in some embodiments, the individual is administered ibrutinib in combination with one or more reversible BTK inhibitors that are not dependent on cysteine 481 for binding. Reversible BTK inhibitors are known in the art and include, but are not limited to, dasatinib, PC-005, RN486, PCI-29732 or terreic acid. In a particular embodiment, the irreversible BTK inhibitor ibrutinib is administered in combination with the reversible BTK inhibitor dasatinib.

In some embodiments, the collection of a sample is performed at a predetermined time or at regular intervals relative to treatment with one or more anticancer agents.

In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of the irreversible BTK inhibitor. In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of ibrutinib to an individual naïve for exposure to ibrutinib. In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of a BTK inhibitor to an individual having CLL. In some embodiments, the sample is obtained 1, 2, 3, 4, 5, 6, 7, 8, 9, 10 times or more over the course of treatment with a BTK inhibitor. In some embodiments, the individual is responsive the treatment with a BTK inhibitor when it is first administered.

In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of the irreversible BTK inhibitor. In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of ibrutinib to an individual naïve for exposure to ibrutinib. In some embodiments, the sample is obtained at 1 week, 2 weeks, 3 weeks, 1 month, 2 months, 3 months, 4 months, 5 months, 6 months, 7 months, 8 months, 9 months, 10 months, 11 months, 12 months, 14 months, 16 months, 18 months, 20 months, 22 months, 24 months, 26 months, 28 months, 30 months, 32 months, 34 months, 36 months or longer following the first administration of ibrutinib to an individual having CLL. In some embodiments, the sample is obtained 1, 2, 3, 4, 5, 6, 7, 8, 9, 10 times or more over the course of treatment with ibrutinib. In some embodiments, the individual is responsive the treatment with ibrutinib when it is first administered.

In some embodiments, the expression level of miR-155 in a sample is compared to the expression level of miR-155 in a control. In some embodiments, the control is a recombinant cell or a population of recombinant cells that express miR-155. Exemplary cell lines include, but are not limited to, Ramos, JY25, CB33, U266, Jurkat, K562, HL60, HDLM2, L428, KMH2, L591, L1236, HEK-293T, OCI-Lyl, OCI-Ly8, and OCI-Ly3. In some embodiments, the expression level of miR-155 in a sample is compared to the expression level of miR-155 in a recombinant cell or a population of recombinant cell in which the cells are from the cell lines Ramos, JY25, CB33, U266, Jurkat, K562, HL60, HDLM2, L428, KMH2, L591, L1236, HEK-293T, OCI-Lyl, OCI-Ly8, and OCI-Ly3.

In some embodiments, the control is a CLL cell or a population of CLL cells. In some embodiments, the expression level of miR-155 in a sample is compared to the expression level of miR-155 in a CLL cell or a population of CLL cells. In some embodiments, the expression level of miR-155 in a sample is compared to the expression level of miR-155 in a CLL cell or a population of CLL cells that are known to be resistant to a BTK inhibitor. In some embodiments, the expression level of miR-155 in a sample is compared to the expression level of miR-155 in a CLL cell or a population of CLL cells that are known to be sensitive to a BTK inhibitor. In some embodiments, the CLL cell line is MEC1, MEC2, WaC3, SeD, B-CLL-LCL, JVM-HH, JVM-2, WR#1, OSU-CLL, WSU-CLL, HG3, I83-E95, I83-LCL, CII, CI, Wa-osel, 232B4, 232A4, PGA1, PG/B95-8, or EHEB. In some embodiments, the expression level of miR-155 in a sample is compared to the expression level of miR-155 in a CLL cell or a population of CLL cells in which the cells are from the CLL cell lines MEC1, MEC2, WaC3, SeD, B-CLL-LCL, JVM-HH, JVM-2, WR#1, OSU-CLL, WSU-CLL, HG3, I83-E95, I83-LCL, CII, CI, Wa-osel, 232B4, 232A4, PGA1, PG/B95-8, or EHEB.

### Biomarkers

Disclosed herein, in certain embodiments, are methods of detecting and determining the presence and/or expression level of biomarkers described herein. In some embodiments, the biomarkers include MiR-155, miR-181a, miR-29c, miR-17-5p, miR-20a, miR-21, miR-92, miR-106a, del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, CCR1, CCR3, CCR4, CCR7, CCR8, CD4, CD26, CD28, CD30, CD81, CD94, CD119, CD183, CD184, CD195, CD212, CD278, c-maf, CRTH2, Gata-3, GM-CSF, IFN γR, IgD, IL-1R, IL-4, IL-5, IL-6, IL-9, IL-10, IL-12β1, IL-13, IL-15, IL-2, IL-12, IL-15, IL-18R, IL-23, IL-27, IL-27R, ST2L/T1, Tim-1, Tim-3, GM-CSF, Granzyme B, IFN-α, IFN-γ, Lymphotoxin, perforin, t-bet, TNF-α, TRANCE, sCD40L, CCL3, and CCL4. In some embodiments, the presence and/or expression level of miR-155 is determined. In some embodiments, the presence and/or expression levels of miR-155 and at least one additional biomarker are determined. In some embodiments, the presence and/or expression levels of miR-155 and at least one of miR-181a, miR-29c, miR-17-5p, miR-20a, miR-21, miR-92, miR-106a, del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, CCR1, CCR3, CCR4, CCR7, CCR8, CD4, CD26, CD28, CD30, CD81, CD94, CD119, CD183, CD184, CD195, CD212, CD278, c-maf, CRTH2, Gata-3, GM-CSF, IFN γR, IgD, IL-1R, IL-4, IL-5, IL-6, IL-9, IL-10, IL-12β1, IL-13, IL-15, IL-2, IL-12, IL-15, IL-18R, IL-23, IL-27, IL-27R, ST2L/T1, Tim-1, Tim-3, GM-CSF, Granzyme B, IFN-α, IFN-γ, Lymphotoxin, perforin, t-bet, TNF-α, TRANCE, sCD40L, CCL3, and CCL4 are determined.

In some embodiments, the presence and/or expression level of miR-155 is used to assess or monitor the efficacy of the treatment with a BTK inhibitor, used to optimize or modify the treatment with a BTK inhibitor, and/or used to assess the responsiveness of the patient having a solid tumor toward the treatment with a BTK inhibitor. In some embodiments, the presence and/or expression levels of miR-155 and at least one additional biomarker are used to assess or monitor the efficacy of the treatment with a BTK inhibitor, used to optimize or modify the treatment with a BTK inhibitor, and/or used to assess the responsiveness of the patient having a solid tumor toward the treatment with a BTK inhibitor. In some embodiments, the presence and/or expression levels of miR-155 and at least one of miR-181a, miR-29c, miR-17-5p, miR-20a, miR-21, miR-92, miR-106a, del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, CCR1, CCR3, CCR4, CCR7, CCR8, CD4, CD26, CD28, CD30, CD81, CD94, CD119, CD183, CD184, CD195, CD212, CD278, c-maf, CRTH2, Gata-3, GM-CSF, IFN γR, IgD, IL-1R, IL-4, IL-5, IL-6, IL-9, IL-10, IL-12β1, IL-13, IL-15, IL-2, IL-12, IL-15, IL-18R, IL-23, IL-27, IL-27R, ST2L/T1, Tim-1, Tim-3, GM-CSF, Granzyme B, IFN-α, IFN-γ, Lymphotoxin, perforin, t-bet, TNF-α, TRANCE, sCD40L, CCL3, and CCL4 are used to assess or monitor the efficacy of the treatment with a BTK inhibitor, used to optimize or modify the treatment with a BTK inhibitor, and/or used to assess the responsiveness of the patient having a solid tumor toward the treatment with a BTK inhibitor.

In some embodiments, the presence and/or expression level of miR-155 is used to assess or monitor the efficacy of the treatment with a BTK inhibitor, used to optimize or modify the treatment with a BTK inhibitor, and/or used to assess the responsiveness of the patient having a hematological malignancy toward the treatment with a BTK inhibitor. In some embodiments, the presence and/or expression levels of miR-155 and at least one additional biomarker are used to assess or monitor the efficacy of the treatment with a BTK inhibitor, used to optimize or modify the treatment with a BTK inhibitor, and/or used to assess the responsiveness of the patient having a hematological malignancy toward the treatment with a BTK inhibitor. In some embodiments, the presence and/or expression levels of miR-155 and at least one of miR-181a, miR-29c, miR-17-5p, miR-20a, miR-21, miR-92, miR-106a, del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, CCR1, CCR3, CCR4, CCR7, CCR8, CD4, CD26, CD28, CD30, CD81, CD94, CD119, CD183, CD184, CD195, CD212, CD278, c-maf, CRTH2, Gata-3, GM-CSF, IFN γR, IgD, IL-1R, IL-4, IL-5, IL-6, IL-9, IL-10, IL-12β1, IL-13, IL-15, IL-2, IL-12, IL-15, IL-18R, IL-23, IL-27, IL-27R, ST2L/T1, Tim-1, Tim-3, GM-CSF, Granzyme B, IFN-α, IFN-γ, Lymphotoxin, perforin, t-bet, TNF-α, TRANCE, sCD40L, CCL3, and CCL4 are used to assess or monitor the efficacy of the treatment with a BTK inhibitor, used to optimize or modify the treatment with a BTK inhibitor, and/or used to assess the responsiveness of the patient having a hematological malignancy toward the treatment with a BTK inhibitor.

In some embodiments, the presence and/or expression level of miR-155 is used to assess or monitor the efficacy of the treatment with a BTK inhibitor, used to optimize or modify the treatment with a BTK inhibitor, and/or used to assess the responsiveness of the patient having CLL toward the treatment with a BTK inhibitor. In some embodiments, the presence and/or expression levels of miR-155 and at least one additional biomarker are used to assess or monitor the efficacy of the treatment with a BTK inhibitor, used to optimize or modify the treatment with a BTK inhibitor, and/or used to assess the responsiveness of the patient having CLL toward the treatment with a BTK inhibitor. In some embodiments, the presence and/or expression levels of miR-155 and at least one of miR-181a, miR-29c, miR-17-5p, miR-20a, miR-21, miR-92, miR-106a, del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, CCR1, CCR3, CCR4, CCR7, CCR8, CD4, CD26, CD28, CD30, CD81, CD94, CD119, CD183, CD184, CD195, CD212, CD278, c-maf, CRTH2, Gata-3, GM-CSF, IFN γR, IgD, IL-1R, IL-4, IL-5, IL-6, IL-9, IL-10, IL-12β1, IL-13, IL-15, IL-2, IL-12, IL-15, IL-18R, IL-23, IL-27, IL-27R, ST2L/T1, Tim-1, Tim-3, GM-CSF, Granzyme B, IFN-α, IFN-γ, Lymphotoxin, perforin, t-bet, TNF-α, TRANCE, sCD40L, CCL3, and CCL4 are used to assess or monitor the efficacy of the treatment with a BTK inhibitor, used to optimize or modify the treatment with a BTK inhibitor, and/or used to assess the responsiveness of the patient having CLL toward the treatment with a BTK inhibitor.

In some embodiments, the BTK inhibitor is selected from among ibrutinib (PCI-32765), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) and LFM-A13. In some embodiments, the BTK inhibitor is ibrutinib.

In some embodiments, the presence and/or expression level of miR-155 is used to assess or monitor the efficacy of the ibrutinib treatment, used to optimize or modify the ibrutinib treatment, and/or used to assess the responsiveness of the patient having a solid tumor toward the ibrutinib treatment. In some embodiments, the presence and/or expression levels of miR-155 and at least one additional biomarker are used to assess or monitor the efficacy of the ibrutinib treatment, used to optimize or modify the ibrutinib treatment, and/or used to assess the responsiveness of the patient having a solid tumor toward the ibrutinib treatment. In some embodiments, the presence and/or expression levels of miR-155 and at least one of miR-181a, miR-29c, miR-17-5p, miR-20a, miR-21, miR-92, miR-106a, del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, CCR1, CCR3, CCR4, CCR7, CCR8, CD4, CD26, CD28, CD30, CD81, CD94, CD119, CD183, CD184, CD195, CD212, CD278, c-maf, CRTH2, Gata-3, GM-CSF, IFN γR, IgD, IL-1R, IL-4, IL-5, IL-6, IL-9, IL-10, IL-12β1, IL-13, IL-15, IL-2, IL-12, IL-15, IL-18R, IL-23, IL-27, IL-27R, ST2L/T1, Tim-1, Tim-3, GM-CSF, Granzyme B, IFN-α, IFN-γ, Lymphotoxin, perforin, t-bet, TNF-α, TRANCE, sCD40L, CCL3, and CCL4 are used to assess or monitor the efficacy of the ibrutinib treatment, used to optimize or modify the ibrutinib treatment, and/or used to assess the responsiveness of the patient having a solid tumor toward the ibrutinib treatment.

In some embodiments, the presence and/or expression level of miR-155 is used to assess or monitor the efficacy of the ibrutinib treatment, used to optimize or modify the ibrutinib treatment, and/or used to assess the responsiveness of the patient having a hematological malignancy toward the ibrutinib treatment. In some embodiments, the presence and/or expression levels of miR-155 and at least one additional biomarker are used to assess or monitor the efficacy of the ibrutinib treatment, used to optimize or modify the ibrutinib treatment, and/or used to assess the responsiveness of the patient having a hematological malignancy toward the ibrutinib treatment. In some embodiments, the presence and/or expression levels of miR-155 and at least one of miR-181a, miR-29c, miR-17-5p, miR-20a, miR-21, miR-92, miR-106a, del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, CCR1, CCR3, CCR4, CCR7, CCR8, CD4, CD26, CD28, CD30, CD81, CD94, CD119, CD183, CD184, CD195, CD212, CD278, c-maf, CRTH2, Gata-3, GM-CSF, IFN γR, IgD, IL-1R, IL-4, IL-5, IL-6, IL-9, IL-10, IL-12β1, IL-13, IL-15, IL-2, IL-12, IL-15, IL-18R, IL-23, IL-27, IL-27R, ST2L/T1, Tim-1, Tim-3, GM-CSF, Granzyme B, IFN-α, IFN-γ, Lymphotoxin, perforin, t-bet, TNF-α, TRANCE, sCD40L, CCL3, and CCL4 are used to assess or monitor the efficacy of the ibrutinib treatment, used to optimize or modify the ibrutinib treatment, and/or used to assess the responsiveness of the patient having a hematological malignancy toward the ibrutinib treatment.

In some embodiments, the presence and/or expression level of miR-155 is used to assess or monitor the efficacy of the ibrutinib treatment, used to optimize or modify the ibrutinib treatment, and/or used to assess the responsiveness of the patient having CLL toward the ibrutinib treatment. In some embodiments, the presence and/or expression levels of miR-155 and at least one additional biomarker are used to assess or monitor the efficacy of the ibrutinib treatment, used to optimize or modify the ibrutinib treatment, and/or used to assess the responsiveness of the patient having CLL toward the ibrutinib treatment. In some embodiments, the presence and/or expression levels of miR-155 and at least one of miR-181a, miR-29c, miR-17-5p, miR-20a, miR-21, miR-92, miR-106a, del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, CCR1, CCR3, CCR4, CCR7, CCR8, CD4, CD26, CD28, CD30, CD81, CD94, CD119, CD183, CD184, CD195, CD212, CD278, c-maf, CRTH2, Gata-3, GM-CSF, IFN γR, IgD, IL-1R, IL-4, IL-5, IL-6, IL-9, IL-10, IL-12β1, IL-13, IL-15, IL-2, IL-12, IL-15, IL-18R, IL-23, IL-27, IL-27R, ST2L/T1, Tim-1, Tim-3, GM-CSF, Granzyme B, IFN-α, IFN-γ, Lymphotoxin, perforin, t-bet, TNF-α, TRANCE, sCD40L, CCL3, and CCL4 are used to assess or monitor the efficacy of the ibrutinib treatment, used to optimize or modify the ibrutinib treatment, and/or used to assess the responsiveness of the patient having CLL toward the ibrutinib treatment.

### Diagnostic Methods

Methods for detecting miRs (e.g., miR-155) and additional biomarkers in an individual are well known in the art (see, for example, Cuneo et al. (1999) Blood 93:1372-1380; Dohner et al. (1997) Blood 89:2516-2522; Butch et al. (2004) Clin. Chem. 50: 2302-2308).

Determining the expression or presence of the biomarkers can be at the protein or nucleic acid level. Where detection is at the protein level, the biomarker protein comprises the full-length polypeptide or any detectable fragment thereof, and can include variants of these protein sequences. Similarly, where detection is at the nucleotide level, the biomarker nucleic acid includes DNA comprising the full-length coding sequence, a fragment of the full-length coding sequence, variants of these sequences, for example naturally occurring variants or splice-variants, or the complement of such a sequence. Biomarker nucleic acids also include RNA, for example, mRNA, comprising the full-length sequence encoding the biomarker protein of interest, a fragment of the full-length RNA sequence of interest, or variants of these sequences. Biomarker proteins and biomarker nucleic acids also include variants of these sequences. By “fragment” is intended a portion of the polynucleotide or a portion of the amino acid sequence and hence protein encoded thereby. Polynucleotides that are fragments of a biomarker nucleotide sequence generally comprise at least 10, 15, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention. “Variant” is intended to mean substantially similar sequences. Generally, variants of a particular biomarker of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that biomarker as determined by sequence alignment programs known in the art.

As provided above, any method known in the art can be used in the methods for determining the expression or presence of biomarker described herein. Circulating levels of biomarkers in a blood sample obtained from a candidate subject, can be measured, for example, by ELISA, radioimmunoassay (RIA), electrochemiluminescence (ECL), Western blot, multiplexing technologies, or other similar methods. Cell surface expression of biomarkers can be measured, for example, by flow cytometry, immunohistochemistry, Western Blot, immunoprecipitation, magnetic bead selection, and quantification of cells expressing either of these cell surface markers. Biomarker RNA expression levels could be measured by RT-PCR, Qt-PCR, microarray, Northern blot, or other similar technologies.

As previously noted, determining the expression or presence of the biomarker of interest at the protein or nucleotide level can be accomplished using any detection method known to those of skill in the art. By “detecting expression” or “detecting the level of” is intended determining the expression level or presence of a biomarker protein or gene in the biological sample. Thus, “detecting expression” encompasses instances where a biomarker is determined not to be expressed, not to be detectably expressed, expressed at a low level, expressed at a normal level, or overexpressed.

In certain aspects of the method provided herein, the one or more subpopulation of lymphocytes are isolated, detected or measured. In certain embodiments, the one or more subpopulation of lymphocytes are isolated, detected or measured using immunophenotyping techniques. In other embodiments, the one or more subpopulation of lymphocytes are isolated, detected or measured using fluorescence activated cell sorting (FACS) techniques.

In certain embodiments of the methods provided herein, the expression level or presence of one or more biomarkers is carried out by a means for nucleic acid amplification, a means for nucleic acid sequencing, a means utilizing a nucleic acid microarray (DNA and RNA), or a means for in situ hybridization using specifically labeled probes.

In other embodiments, the determining the expression or presence of one or more biomarkers is carried out through gel electrophoresis. In one embodiment, the determination is carried out through transfer to a membrane and hybridization with a specific probe.

In other embodiments, the determining the expression or presence of one or more biomarkers carried out by a diagnostic imaging technique.

In still other embodiments, the determining the expression or presence of one or more biomarkers carried out by a detectable solid substrate. In one embodiment, the detectable solid substrate is paramagnetic nanoparticles functionalized with antibodies.

Methods for detecting expression of the biomarkers described herein, within the test and control biological samples comprise any methods that determine the quantity or the presence of these markers either at the nucleic acid or protein level. Such methods are well known in the art and include but are not limited to western blots, northern blots, ELISA, immunoprecipitation, immunofluorescence, flow cytometry, immunohistochemistry, nucleic acid hybridization techniques, nucleic acid reverse transcription methods, and nucleic acid amplification methods. In particular embodiments, expression of a biomarker is detected on a protein level using, for example, antibodies that are directed against specific biomarker proteins. These antibodies can be used in various methods such as Western blot, ELISA, multiplexing technologies, immunoprecipitation, or immunohistochemistry techniques.

Any means for specifically identifying and quantifying a biomarker (for example, biomarker, a biomarker of cell survival or proliferation, a biomarker of apoptosis, a biomarker of a Btk-mediated signaling pathway) in the biological sample of a candidate subject is contemplated. In some embodiments, the expression or presence of one or more of the biomarkers described herein are determined at the nucleic acid level. Nucleic acid-based techniques for assessing expression are well known in the art and include, for example, determining the level of biomarker mRNA in a biological sample. Many expression detection methods use isolated RNA. Any RNA isolation technique that does not select against the isolation of mRNA can be utilized for the purification of RNA (see, e.g., Ausubel et al., ed. (1987-1999) Current Protocols in Molecular Biology (John Wiley & Sons, New York). Additionally, large numbers of tissue samples can readily be processed using techniques well known to those of skill in the art, such as, for example, the single-step RNA isolation process disclosed in U.S. Pat. No. 4,843,155.

In some embodiments, the detection of a biomarker or other protein of interest is assayed at the nucleic acid level using nucleic acid probes. The term “nucleic acid probe” refers to any molecule that is capable of selectively binding to a specifically intended target nucleic acid molecule, for example, a nucleotide transcript. Probes can be synthesized by one of skill in the art, or derived from appropriate biological preparations. In some embodiments, probes are specifically designed to be labeled, for example, with a radioactive label, a fluorescent label, an enzyme, a chemiluminescent tag, a colorimetric tag, or other labels or tags that are discussed above or that are known in the art. Examples of molecules that can be utilized as probes include, but are not limited to, RNA and DNA.

For example, isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding a biomarker, biomarker described herein above. Hybridization of an mRNA with the probe indicates that the biomarker or other target protein of interest is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in a gene chip array. A skilled artisan can readily adapt known mRNA detection methods for use in detecting the level of mRNA encoding the biomarkers or other proteins of interest.

An alternative method for determining the level of an mRNA of interest in a sample involves the process of nucleic acid amplification, e.g., by RT-PCR (see, for example, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System).

In some embodiments, expression levels of an RNA of interest are monitored using a membrane blot (such as used in hybridization analysis such as Northern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934. In some embodiments, the detection of expression also comprises using nucleic acid probes in solution.

In one embodiment of the invention, microarrays are used to determine expression or presence of one or more biomarkers. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316. High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261. Although a planar array surface is preferred, in some embodiments, the array is fabricated on a surface of virtually any shape or even a multiplicity of surfaces. In some embodiments, arrays are peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. In some embodiments, arrays are packaged in such a manner as to allow for diagnostics or other manipulation of an all-inclusive device. See, for example, U.S. Pat. Nos. 5,856,174 and 5,922,591.

In some embodiments, expression level of a biomarker protein of interest in a biological sample is detected by means of a binding protein capable of interacting specifically with that biomarker protein or a biologically active variant thereof. In some embodiments, labeled antibodies, binding portions thereof, or other binding partners are used. The word “label” when used herein refers to a detectable compound or composition that is conjugated directly or indirectly to the antibody so as to generate a “labeled” antibody. In some embodiments, the label is detectable by itself (e.g., radioisotope labels or fluorescent labels) or, in the case of an enzymatic label, catalyzes chemical alteration of a substrate compound or composition that is detectable.

In some embodiments, the antibodies for detection of a biomarker protein are monoclonal or polyclonal in origin, or are synthetically or recombinantly produced. The amount of complexed protein, for example, the amount of biomarker protein associated with the binding protein, for example, an antibody that specifically binds to the biomarker protein, is determined using standard protein detection methodologies known to those of skill in the art. A detailed review of immunological assay design, theory and protocols can be found in numerous texts in the art (see, for example, Ausubel et al., eds. (1995) Current Protocols in Molecular Biology) (Greene Publishing and Wiley-Interscience, NY)); Coligan et al., eds. (1994) Current Protocols in Immunology (John Wiley & Sons, Inc., New York, N.Y.).

The choice of marker used to label the antibodies will vary depending upon the application. However, the choice of the marker is readily determinable to one skilled in the art. In some embodiments, these labeled antibodies are used in immunoassays as well as in histological applications to detect the presence of any biomarker or protein of interest. In some embodiments, the labeled antibodies are polyclonal or monoclonal. Further, in some embodiments, the antibodies for use in detecting a protein of interest are labeled with a radioactive atom, an enzyme, a chromophoric or fluorescent moiety, or a colorimetric tag as described elsewhere herein. The choice of tagging label also will depend on the detection limitations desired. Enzyme assays (ELISAs) typically allow detection of a colored product formed by interaction of the enzyme-tagged complex with an enzyme substrate. Radionuclides that can serve as detectable labels include, for example, 1-131, 1-123, 1-125, Y-90, Re-188, Re-186, At-211, Cu-67, Bi-212, and Pd-109. Examples of enzymes that can serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. In some embodiments, the antibodies are conjugated to these labels by methods known in the art. For example, in some embodiments, enzymes and chromophoric molecules are conjugated to the antibodies by means of coupling agents, such as dialdehydes, carbodiimides, dimaleimides, and the like. Alternatively, in some embodiments, conjugation occurs through a ligand-receptor pair. Examples of suitable ligand-receptor pairs are biotin-avidin or biotin-streptavidin, and antibody-antigen.

In certain embodiments, expression or presence of one or more biomarkers or other proteins of interest within a biological sample, for example, a sample of bodily fluid, is determined by radioimmunoassays or enzyme-linked immunoassays (ELISAs), competitive binding enzyme-linked immunoassays, dot blot (see, for example, Promega Protocols and Applications Guide (2^{nd} ed.; Promega Corporation (1991), Western blot (see, for example, Sambrook et al. (1989) Molecular Cloning, A Laboratory Manual, Vol. 3, Chapter 18 (Cold Spring Harbor Laboratory Press, Plainview, N.Y.), chromatography, preferably high performance liquid chromatography (HPLC), or other assays known in the art. Thus, in some embodiments, the detection assays involve steps such as, but not limited to, immunoblotting, immunodiffusion, immunoelectrophoresis, or immunoprecipitation.

### Btk Inhibitor Compounds Including Ibrutinib and Pharmaceutically Acceptable Salts Thereof

An exemplary Btk inhibitor compound described herein (e.g., Ibrutinib) is selective for Btk and kinases having a cysteine residue in an amino acid sequence position of the tyrosine kinase that is homologous to the amino acid sequence position of cysteine 481 in Btk. The Btk inhibitor compound can form a covalent bond with Cys 481 of Btk (e.g., via a Michael reaction).

In some embodiments, the Btk inhibitor is a compound of Formula (A) having the structure:

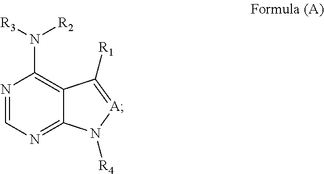

wherein:

A is N;

R₁ is phenyl-O-phenyl or phenyl-S-phenyl;

R₂ and R₃ are independently H;

R₄ is L₃-X-L₄-G, wherein,

L₃ is optional, and when present is a bond, optionally substituted or unsubstituted alkyl, optionally substituted or unsubstituted cycloalkyl, optionally substituted or unsubstituted alkenyl, optionally substituted or unsubstituted alkynyl;

X is optional, and when present is a bond, —O—, —C(═O)—, —S—, —S(═O)—, —S(═O)₂—, —NH—, —NR₉—, —NHC(O)—, —C(O)NH—, —NR₉C(O)—, —C(O)NR₉—, —S(═O)₂NH—, —NHS(═O)₂—, —S(═O)₂NR₉—, —NR₉S(═O)₂—, —OC(O)NH—, —NHC(O)O—, —OC(O)NR₉—, —NR₉C(O)O—, —CH═NO—, —ON═CH—, —NR₁₀C(O)NR₁₀—, heteroaryl-, aryl-, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;

L₄ is optional, and when present is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycle;

or L₃, X and L₄ taken together form a nitrogen containing heterocyclic ring;

G is

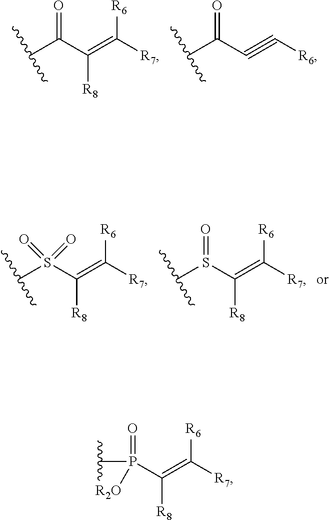

wherein,

R₆, R₇ and R₈ are independently selected from among H, halogen, CN, OH, substituted or unsubstituted alkyl or substituted or unsubstituted heteroalkyl or substituted or unsubstituted cycloalkyl, substituted or unsubstituted heterocycloalkyl, substituted or unsubstituted aryl, substituted or unsubstituted heteroaryl;

each R₉ is independently selected from among H, substituted or unsubstituted lower alkyl, and substituted or unsubstituted lower cycloalkyl;

each R₁₀ is independently H, substituted or unsubstituted lower alkyl, or substituted or unsubstituted lower cycloalkyl; or

two R₁₀ groups can together form a 5-, 6-, 7-, or 8-membered heterocyclic ring; or

R₁₀ and R₁₁ can together form a 5-, 6-, 7-, or 8-membered heterocyclic ring; or each R₁₁ is independently selected from H or substituted or unsubstituted alkyl; or a pharmaceutically acceptable salt thereof. In some embodiments, L₃, X and L₄ taken together form a nitrogen containing heterocyclic ring. In some embodiments, the nitrogen containing heterocyclic ring is a piperidine group. In some embodiments, G is

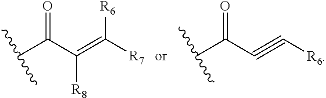

In some embodiments, the compound of Formula (A) is 1-[(3R)-3-[4-amino-3-(4-phenoxyphenyl)pyrazolo[3,4-d]pyrimidin-1-yl]piperidin-1-yl]prop-2-en-1-one.

“Ibrutinib” or “1-((R)-3-(4-amino-3-(4-phenoxyphenyl)-1H-pyrazolo[3,4-d]pyrimidin-1-yl)piperidin-1-yl)prop-2-en-1-one” or “1-{(3R)-3-[4-amino-3-(4-phenoxyphenyl)-1H-pyrazolo[3,4-d]pyrimidin-1-yl]piperidin-1-yl}prop-2-en-1-one” or “2-Propen-1-one, 1-[(3R)-3-[4-amino-3-(4-phenoxyphenyl)-1H-pyrazolo[3,4-d]pyrimidin-1-yl]-1-piperidinyl-” or Ibrutinib or any other suitable name refers to the compound with the following structure:

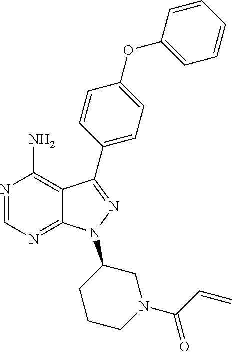 [SMILES: C=CC(=O)N1CCC[C@@H](n2nc(-c3ccc(Oc4ccccc4)cc3)c3c(N)ncnc32)C1]

A wide variety of pharmaceutically acceptable salts is formed from Ibrutinib and includes:
acid addition salts formed by reacting Ibrutinib with an organic acid, which includes aliphatic mono- and dicarboxylic acids, phenyl-substituted alkanoic acids, hydroxyl alkanoic acids, alkanedioic acids, aromatic acids, aliphatic and aromatic sulfonic acids, amino acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like;
acid addition salts formed by reacting Ibrutinib with an inorganic acid, which includes hydrochloric acid, hydrobromic acid, sulfuric acid, nitric acid, phosphoric acid, hydroiodic acid, hydrofluoric acid, phosphorous acid, and the like.

The term “pharmaceutically acceptable salts” in reference to Ibrutinib refers to a salt of Ibrutinib, which does not cause significant irritation to a mammal to which it is administered and does not substantially abrogate the biological activity and properties of the compound.

It should be understood that a reference to a pharmaceutically acceptable salt includes the solvent addition forms (solvates). Solvates contain either stoichiometric or non-stoichiometric amounts of a solvent, and are formed during the process of product formation or isolation with pharmaceutically acceptable solvents such as water, ethanol, methanol, methyl tert-butyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In one aspect, solvates are formed using, but limited to, Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C(R3), (November 2005). Hydrates are formed when the solvent is water, or alcoholates are formed when the solvent is alcohol. In some embodiments, solvates of Ibrutinib, or pharmaceutically acceptable salts thereof, are conveniently prepared or formed during the processes described herein. In some embodiments, solvates of Ibrutinib are anhydrous. In some embodiments, Ibrutinib, or pharmaceutically acceptable salts thereof, exist in unsolvated form. In some embodiments, Ibrutinib, or pharmaceutically acceptable salts thereof, exist in unsolvated form and are anhydrous.

In yet other embodiments, Ibrutinib, or a pharmaceutically acceptable salt thereof, is prepared in various forms, including but not limited to, amorphous phase, crystalline forms, milled forms and nano-particulate forms. In some embodiments, Ibrutinib, or a pharmaceutically acceptable salt thereof, is amorphous. In some embodiments, Ibrutinib, or a pharmaceutically acceptable salt thereof, is amorphous and anhydrous. In some embodiments, Ibrutinib, or a pharmaceutically acceptable salt thereof, is crystalline. In some embodiments, Ibrutinib, or a pharmaceutically acceptable salt thereof, is crystalline and anhydrous.

In some embodiments, Ibrutinib is prepared as outlined in U.S. Pat. No. 7,514,444.

In one aspect are compounds having the structure of Formula (A2-A6):

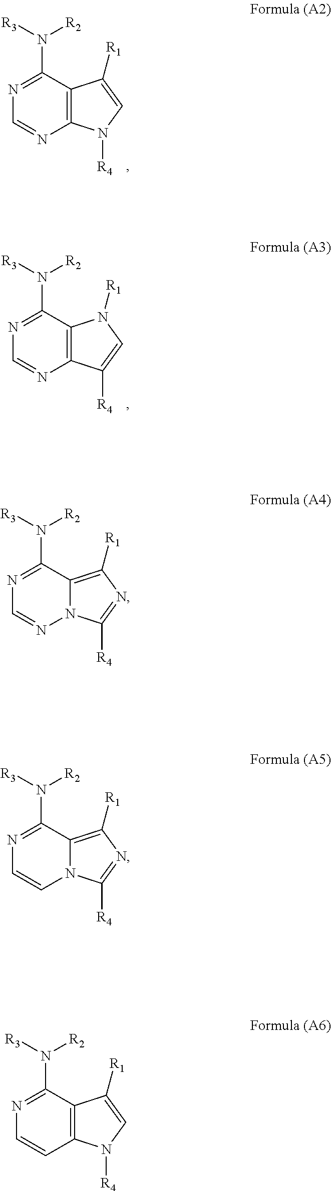

wherein
R₁ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycle), L₂-(substituted or unsubstituted heteroaryl), or L₂-(substituted or unsubstituted aryl), where L₂ is a bond, O, S, —S(═O), —S(═O)₂, C(═O), -(substituted or unsubstituted C₁-C₆ alkylene), or -(substituted or unsubstituted C₂-C₆ alkenylene);
R₂ and R₃ are independently selected from H, lower alkyl and substituted lower alkyl;
R₄ is L₃-X-L₄-G, wherein,
   L₃ is optional, and when present is a bond, optionally substituted or unsubstituted alkylene, optionally substituted or unsubstituted cycloalkylene, optionally substituted or unsubstituted alkenylene, optionally substituted or unsubstituted alkynylene;
   X is optional, and when present is a bond, O, —C(═O), S, —S(═O), —S(═O)₂, —NH, —NR₉, —NHC(O), —C(O)NH, —NR₉C(O), —C(O)NR₉, —S(═O)₂NH, —NHS(═O)₂, —S(═O)₂NR₉—, —NR₉S(═O)₂, —OC(O)NH—, —NHC(O)O—, —OC(O)NR₉—, —NR₉C(O)O—, —CH═NO—, —ON═CH—, —NR₁₀C(O)NR₁₀—, heteroarylene, arylene, —NR₁₀C(═NR₁₁)NR₁₀—, —NR₁₀C(═NR₁₁)—, —C(═NR₁₁)NR₁₀—, —OC(═NR₁₁)—, or —C(═NR₁₁)O—;
   L₄ is optional, and when present is a bond, substituted or unsubstituted alkylene, substituted or unsubstituted cycloalkylene, substituted or unsubstituted alkenylene, substituted or unsubstituted alkynylene, substituted or unsubstituted arylene, substituted or unsubstituted heteroarylene, substituted or unsubstituted heterocyclene;
   or L₃, X and L₄ taken together form a nitrogen containing heterocyclic ring;
   G is

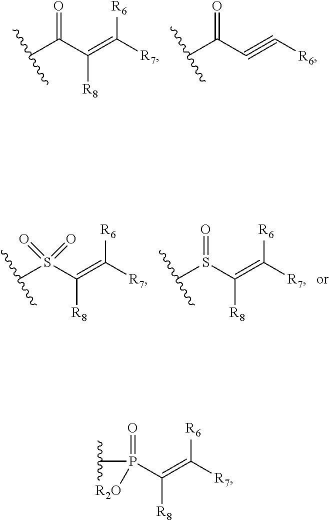

wherein, R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₉ is selected from among H, substituted or unsubstituted lower alkyl, and substituted or unsubstituted lower cycloalkyl;
each R₁₀ is independently H, substituted or unsubstituted lower alkyl, or substituted or unsubstituted lower cycloalkyl; or
two R₁₀ groups can together form a 5-, 6-, 7-, or 8-membered heterocyclic ring; or
R₁₀ and R₁₁ can together form a 5-, 6-, 7-, or 8-membered heterocyclic ring; or
R₁₁ is selected from H, —S(═O)₂R₈, —S(═O)₂NH₂, —C(O)R₈, —CN, —NO₂, heteroaryl, or heteroalkyl; and pharmaceutically active metabolites, or pharmaceutically acceptable solvates, pharmaceutically acceptable salts, or pharmaceutically acceptable prodrugs thereof.

In a further or alternative embodiment, the compound of Formula (A2-A6) has the following structure of Formula (B2-B6):

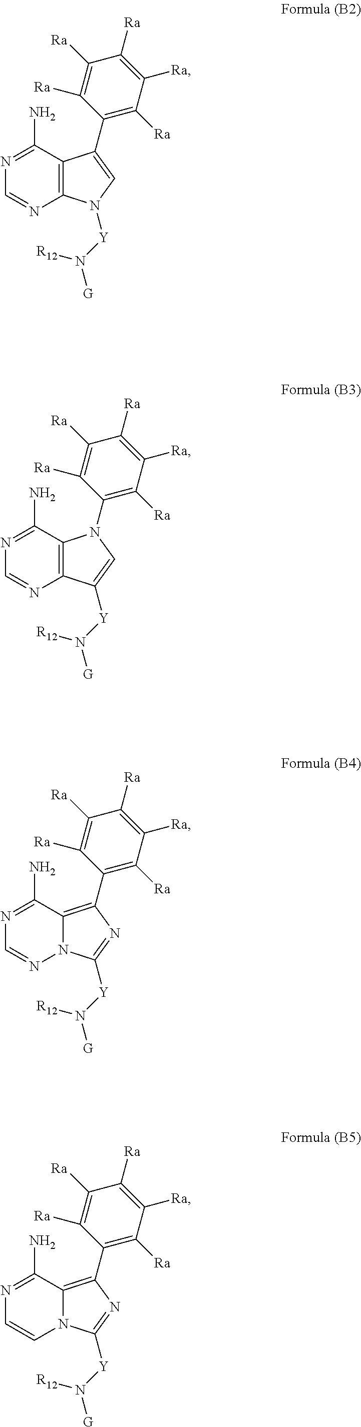

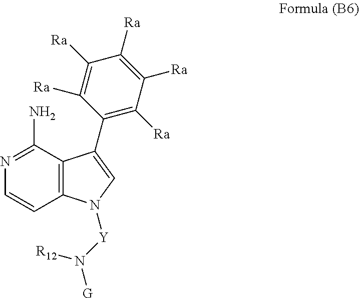

wherein:
Y is alkylene or substituted alkylene, or a 4-, 5-, or 6-membered cycloalkylene ring;
each Rₐ is independently H, halogen, —CF₃, —CN, —NO₂, OH, NH₂, -Lₐ-(substituted or unsubstituted alkyl), -Lₐ-(substituted or unsubstituted alkenyl), -Lₐ-(substituted or unsubstituted heteroaryl), or -Lₐ-(substituted or unsubstituted aryl), wherein Lₐ is a bond, O, S, —S(═O), —S(═O)₂, NH, C(O), CH₂, —NHC(O)O, —NHC(O), or —C(O)NH;
G is

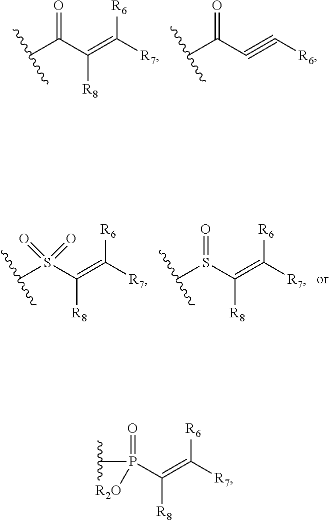

wherein,
R₆, R₇ and R₈ are independently selected from among H, lower alkyl or substituted lower alkyl, lower heteroalkyl or substituted lower heteroalkyl, substituted or unsubstituted lower cycloalkyl, and substituted or unsubstituted lower heterocycloalkyl;
R₁₂ is H or lower alkyl; or
Y and R₁₂ taken together form a 4-, 5-, or 6-membered heterocyclic ring; and
pharmaceutically acceptable active metabolites, pharmaceutically acceptable solvates, pharmaceutically acceptable salts, or pharmaceutically acceptable prodrugs thereof.

In further or alternative embodiments, G is selected from among

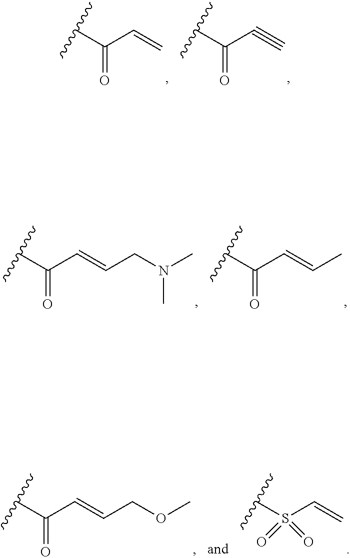

In further or alternative embodiments,

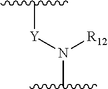

is selected from among

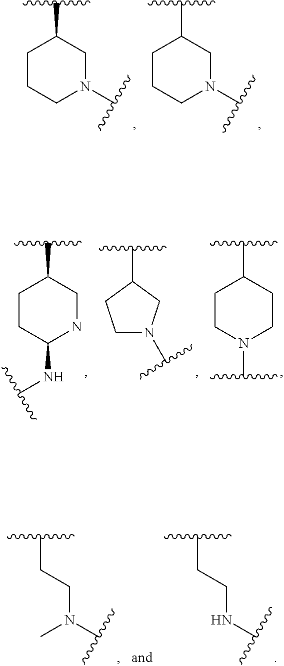

In a further or alternative embodiment, the “G” group of any of Formula (A2-A6) or Formula (B2-B6) is any group that is used to tailor the physical and biological properties of the molecule. Such tailoring/modifications are achieved using groups which modulate Michael acceptor chemical reactivity, acidity, basicity, lipophilicity, solubility and other physical properties of the molecule. The physical and biological properties modulated by such modifications to G include, by way of example only, enhancing chemical reactivity of Michael acceptor group, solubility, in vivo absorption, and in vivo metabolism. In addition, in vivo metabolism includes, by way of example only, controlling in vivo PK properties, off-target activities, potential toxicities associated with cypP450 interactions, drug-drug interactions, and the like. Further, modifications to G allow for the tailoring of the in vivo efficacy of the compound through the modulation of, by way of example, specific and non-specific protein binding to plasma proteins and lipids and tissue distribution in vivo.

In some embodiments, the Btk inhibitor is PCI-45292, PCI-45466, ACP-196 (Acerta Pharma BV), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK417891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), or HM71224 (Hanmi Pharmaceutical Company Limited).

In some embodiments, the Btk inhibitor is 4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide (CGI-1746); 7-benzyl-1-(3-(piperidin-1-yl)propyl)-2-(4-(pyridin-4-yl)phenyl)-1H-imidazo[4,5-g]quinoxalin-6(5H)-one (CTA-056); (R)—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide (GDC-0834); 6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one (RN-486); N-[5-[5-(4-acetylpiperazine-1-carbonyl)-4-methoxy-2-methylphenyl]sulfanyl-1,3-thiazol-2-yl]-4-[(3,3-dimethylbutan-2-ylamino)methyl]benzamide (BMS-509744, HY-11092); or N-(5-((5-(4-Acetylpiperazine-1-carbonyl)-4-methoxy-2-methylphenyl)thio)thiazol-2-yl)-4-(((3-methylbutan-2-yl)amino)methyl)benzamide (HY11066); or a pharmaceutically acceptable salt thereof.

In some embodiments, the Btk inhibitor is:

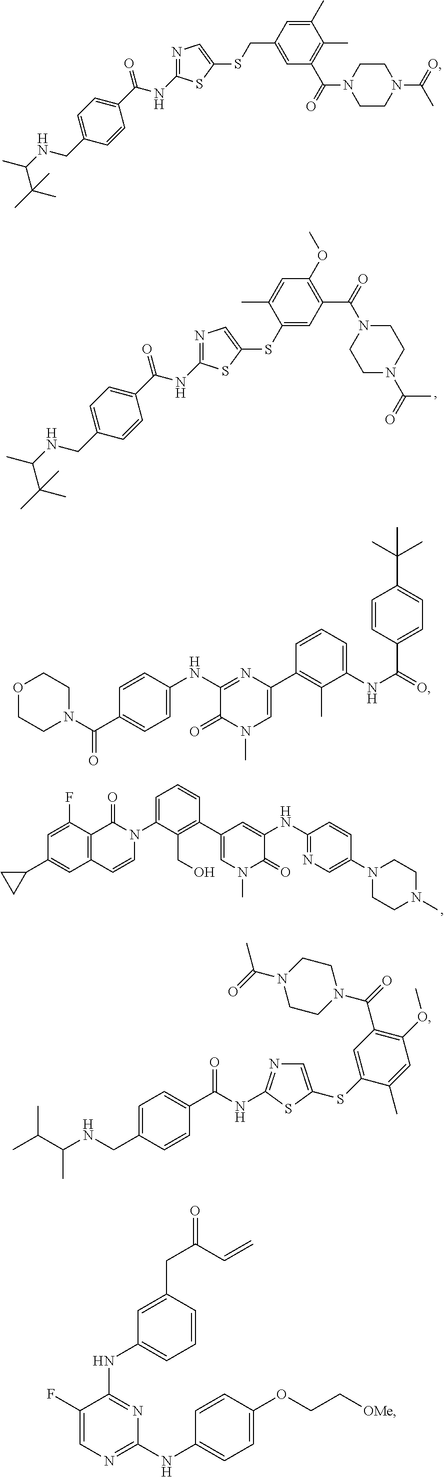

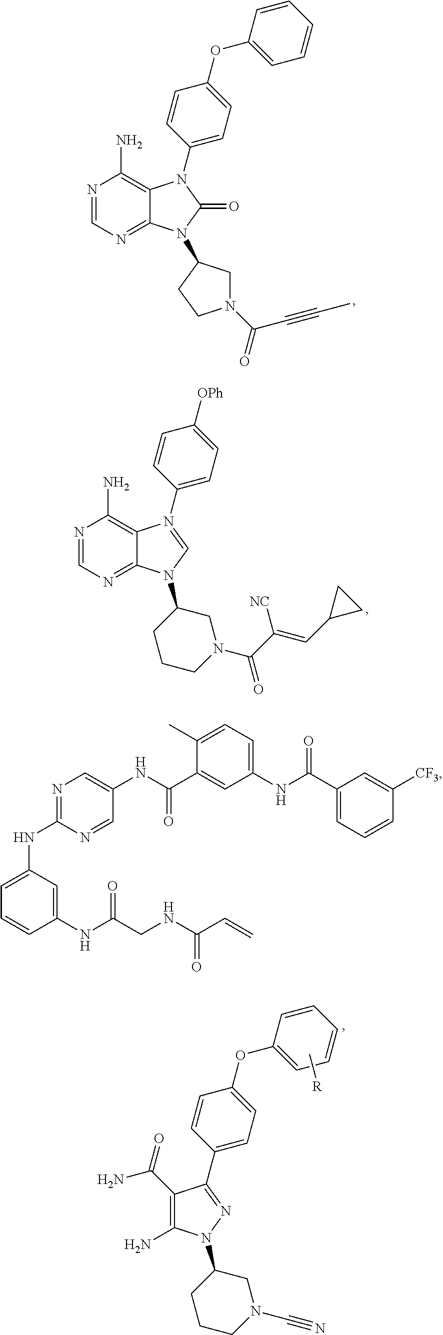

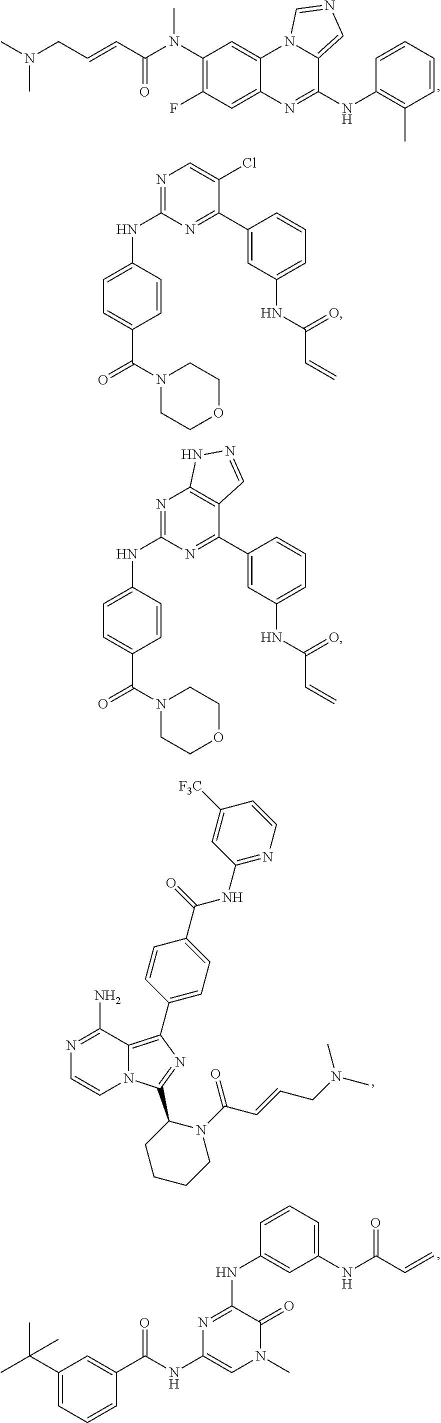

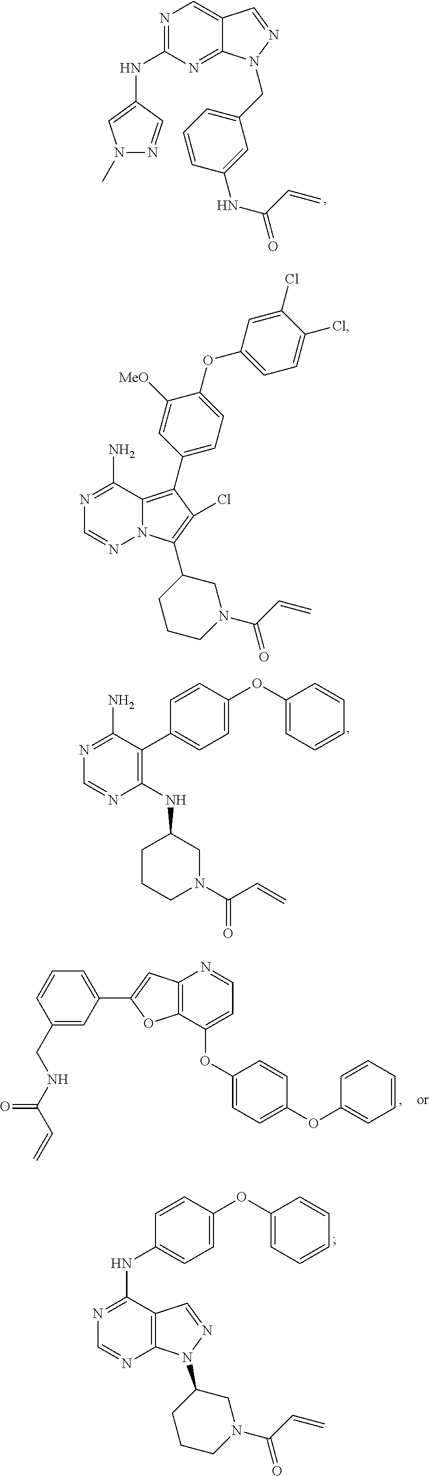

or a pharmaceutically acceptable salt thereof.

### Dosing and Treatment Regimens

Disclosed herein, in certain embodiments, are methods of treating an individual having CLL, based on the expression level of miR-155 in a sample from the individual following administration of a BTK inhibitor-based treatment (e.g., an ibrutinib-based treatment); and optimize or modify the treatment if the expression level of miR-155 is decreased by a predetermined amount relative to the expression level of miR-155 prior to the treatment. Also disclosed herein, in certain embodiments, are methods of treating an individual having a solid tumor, based on the expression level of miR-155 in a sample from the individual following administration of a BTK inhibitor-based treatment (e.g., an ibrutinib-based treatment); and optimize or modify the treatment if the expression level of miR-155 is decreased by a predetermined amount relative to the expression level of miR-155 prior to the treatment. Further disclosed herein, in certain embodiments, are methods of treating an individual having a hematological malignancy, based on the expression level of miR-155 in a sample from the individual following administration of a BTK inhibitor-based treatment (e.g., an ibrutinib-based treatment); and optimize or modify the treatment if the expression level of miR-155 is decreased by a predetermined amount relative to the expression level of miR-155 prior to the treatment.

In some embodiments, the treatment regimen is continued. In some embodiments, the treatment regimen is modified. In some embodiments, the dosage of the BTK inhibitor is increased. In some embodiments, the dosage of the BTK inhibitor is decreased. In some embodiments, the dosage of the BTK inhibitor is not modified. In some embodiments, the frequency of administration of the BTK inhibitor is increased. In some embodiments, the frequency of administration of the BTK inhibitor is decreased. In some embodiments, the frequency of administration of the BTK inhibitor is not modified. In some embodiments, the timing of administration of the BTK inhibitor is modified (e.g., time of day or time relative to administration of other therapeutic agents). In some embodiments, the timing of administration of the BTK inhibitor is not modified. In some embodiments, an additional therapeutic agent is administered. In some embodiments, an additional anticancer agent is administered. In some embodiments, the therapy is a maintenance therapy.

In some embodiments, the individual is monitored every month, every 2 months, every 3 months, every 4 months, every 5 months, every 6 months, every 7 months, every 8 months, every 9 months, every 10 months, every 11 months, or every year to determine the level of expression of miR-155.

In some embodiments, the therapy comprises multiple cycles of administration of a BTK inhibitor. In some embodiments, a cycle of administration is one month, 2 months, 3 months, 4 months, 6 months, 6 months, 7 months, 8 months, 9 months, 10 months, 11 months, 12 months or longer. In some embodiments, a cycle of administration comprises administration of a single therapeutic dosage of a BTK inhibitor over the cycle. In some embodiments, a cycle of administration comprises two or more different dosages of a BTK inhibitor over the cycle. In some embodiments, the dosage of a BTK inhibitor differs over consecutive cycles. In some embodiments, the dosage of a BTK inhibitor increases over consecutive cycles. In some embodiments, the dosage of a BTK inhibitor is the same over consecutive cycles.

In some embodiments, the therapy comprises administration of a daily dosage of a BTK inhibitor. In some embodiments, the daily dosage of ibrutinib administered is at or about 10 mg per day to about 2000 mg per day, such as for example, about 50 mg per day to about 1500 mg per day, such as for example about 100 mg per day to about 1000 mg per day, such as for example about 250 mg per day to about 850 mg per day, such as for example about 300 mg per day to about 600 mg per day. In a particular embodiment, the dosage of a BTK inhibitor is about 840 mg per day. In a particular embodiment, the dosage of a BTK inhibitor is about 560 mg per day. In a particular embodiment, the dosage of a BTK inhibitor is about 420 mg per day. In a particular embodiment, the dosage of a BTK inhibitor is about 140 mg per day.

In some embodiments, the therapy comprises administration of a daily dosage of ibrutinib. In some embodiments, the daily dosage of ibrutinib administered is at or about 10 mg per day to about 2000 mg per day, such as for example, about 50 mg per day to about 1500 mg per day, such as for example about 100 mg per day to about 1000 mg per day, such as for example about 250 mg per day to about 850 mg per day, such as for example about 300 mg per day to about 600 mg per day. In a particular embodiment, the dosage of ibrutinib is about 840 mg per day. In a particular embodiment, the dosage of ibrutinib is about 560 mg per day. In a particular embodiment, the dosage of ibrutinib is about 420 mg per day. In a particular embodiment, the dosage of ibrutinib is about 140 mg per day.

In some embodiments, a BTK inhibitor is administered once per day, two times per day, three times per day or more frequent. In a particular embodiment, a BTK inhibitor is administered once per day.

In some embodiments, ibrutinib is administered once per day, two times per day, three times per day or more frequent. In a particular embodiment, ibrutinib is administered once per day.

In some embodiments, the dosage of a BTK inhibitor is escalated over time. In some embodiments, the dosage of a BTK inhibitor is escalated from at or about 1.25 mg/kg/day to at or about 12.5 mg/kg/day over a predetermined period of time. In some embodiments the predetermined period of time is over 1 month, over 2 months, over 3 months, over 4 months, over 5 months, over 6 months, over 7 months, over 8 months, over 9 months, over 10 months, over 11 months, over 12 months, over 18 months, over 24 months or longer.

In some embodiments, the dosage of ibrutinib is escalated over time. In some embodiments, the dosage of ibrutinib is escalated from at or about 1.25 mg/kg/day to at or about 12.5 mg/kg/day over a predetermined period of time. In some embodiments the predetermined period of time is over 1 month, over 2 months, over 3 months, over 4 months, over 5 months, over 6 months, over 7 months, over 8 months, over 9 months, over 10 months, over 11 months, over 12 months, over 18 months, over 24 months or longer.

In some embodiments, a cycle of administration comprises administration of a BTK inhibitor in combination with an additional therapeutic agent. In some embodiments the additional therapeutic is administered simultaneously, sequentially, or intermittently with a BTK inhibitor. In some embodiments the additional therapeutic agent is an anticancer agent. In some embodiments, the additional therapeutic agent is an anticancer agent for the treatment of CLL. Exemplary anti-cancer agents for administration in a combination with a BTK inhibitor are provided elsewhere herein. In a particular embodiment, the anticancer agent is rituximab. In a particular embodiment, the anticancer agent is fludarabine. In a particular embodiment, the anticancer agent is ofatumumab. In some embodiments, the additional anti-cancer agent is a reversible BTK inhibitor.

In some embodiments, a cycle of administration comprises administration of ibrutinib in combination with an additional therapeutic agent. In some embodiments the additional therapeutic is administered simultaneously, sequentially, or intermittently with ibrutinib. In some embodiments the additional therapeutic agent is an anticancer agent. In some embodiments the additional therapeutic agent is an anti-cancer agent for the treatment of CLL. Exemplary anticancer agents for administration in a combination with ibrutinib are provided elsewhere herein. In a particular embodiment, the anticancer agent is fludarabine. In a particular embodiment, the anticancer agent is ofatumumab. In some embodiments, the additional anti-cancer agent is a reversible BTK inhibitor.

### Kits and Articles of Manufacture

For use in the diagnostic and therapeutic applications described herein, kits and articles of manufacture are also described herein. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from any acceptable material including, e.g., glass or plastic.

In some embodiments, the kits provided herein are for use in determining the expression level of miR-155.

In some embodiments, the kits provided herein are for use as a companion diagnostic with a BTK inhibitor. In some embodiments the kits are employed for selecting patients for treatment with a BTK inhibitor, for identifying individuals as sensitive to a BTK inhibitor of for evaluating treatment with a BTK inhibitor. In some embodiments the kits are employed for selecting patients for treatment with a BTK inhibitor, for identifying an individual who has relapsed or likely to have a relapse to a BTK inhibitor, for monitoring the progression of a solid tumor or a hematological malignancy such as CLL to a BTK inhibitor, or combinations thereof.

In some embodiments, the kits provided herein are for use as a companion diagnostic with ibrutinib. In some embodiments the kits are employed for selecting patients for treatment with ibrutinib, for identifying individuals as sensitive to ibrutinib of for evaluating treatment with ibrutinib. In some embodiments the kits are employed for selecting patients for treatment with ibrutinib, for identifying an individual who has relapsed or likely to have a relapse to ibrutinib, for monitoring the progression of a solid tumor or a hematological malignancy such as CLL to ibrutinib, or combinations thereof.

The kits provided herein contain one or more reagents for the detection of miR-155 expression. Exemplary reagents include but are not limited to, antibodies, buffers, nucleic acids, microarrays, ELISA plates, substrates for enzymatic staining, chromagens or other materials, such as slides, containers, microtiter plates, and optionally, instructions for performing the methods. Those of skill in the art will recognize many other possible containers and plates and reagents that can be used for contacting the various materials

### EXAMPLES

These examples are provided for illustrative purposes only and not to limit the scope of the claims provided herein.

### Example 1

### Materials

Samples examined were derived from cryo-preserved samples from CLL patients enrolled on chemoimmunotherapy trials CALGB 9712 and CALGB 10101 (see, Byrd et al. “Randomized phase 2 study of fludarabine with concurrent versus sequential treatment with rituximab in symptomatic, untreated patients with B-cell chronic lymphocytic leukemia: results from Cancer and Leukemia Group B 9712 (CALGB 9712),” Blood 101:6-14 (2003); Lin et al., “Consolidation therapy with subcutaneous alemtuzumab after fludarabine and rituximab induction therapy for previously untreated chronic lymphocytic leukemia: final analysis of CALGB 10101,” J Clin Oncol 28:4500-4506 (2010)) Protocols were approved by IRB and patients provided written informed consent prior to participation.

A second set of samples were obtained from CLL patients enrolled in OSU-10053 and OSU-10053 (NCT01589302) (see, Jaglowski et al. “A phase ib/ii study evaluating activity and tolerability of Btk inhibitor PCI-32765 and ofatumumab in patients with chronic lymphocytic leukemia/small lymphocytic lymphoma (CLL/SLL) and related disease,” J clin oncol 30, 2012 (suppl; abstr 6508); Maddocks et al., “A phase 2 study of the BTK inhibitor ibrutinib in genetic risk-stratifed relapsed and refractory patients with chronic lymphocytic leukemia (CLL)/small lymphocytic lymphoma (SLL). EHA 2014 (abstr S1342)) In addition, samples were obtained at 1 year (C12D1) and time of response as well as progression in specific groups of interest.

miRNA Analysis

RNA was extracted using Trizol and purified with the miRVana kit (Ambion) according to the manufacturer's protocol. miR analysis was performed by NanoString Technologies' nCounter platform. Serial samples from ibrutinib treated (420 mg/day) patients were obtained pre-treatment, day 8, and day 29 of therapy on OSU-10053 and pre-treatment and day 29 on

Fisher's exact test and the non-parametric Wilcoxon rank sum test were used to test associations between high and low expressers of miR-155 (dichotomized using the median expression value) and categorical or continuous variables, respectively. Progression-free survival and overall survival for high and low expressers who had received chemoimmunotherapy were described with the Kaplan-Meier method and the log-rank test was used to test differences between curves. To test the association between miR-155 expression and time to event endpoints when adjusting for other prognostic factors, multivariable proportional hazard models were fit using backward selection and α=0.05. Covariates considered for model selection included age, sex, hemoglobin, white blood cell count, Rai stage, performance status, and high-risk cytogenetics (del(17p)/del(11q) versus other). All models controlled for treatment study. Departures in the proportional hazards assumption of miR-155 expression on overall survival was identified, and all modeling for this endpoint included a time-dependent covariate that allowed the risk of death to be different prior to and after a time on study of 4 years.

RNA was isolated and quantitative RT-PCR expression was performed using Taqman miRNA assay (Applied Biosystems). The miR-155 expression was normalized to housekeeping gene RNU44 using the 2^{-ΔCT} method and the negative ΔCT values were used in all analyses (i.e. log-transformed (base 2) expression values). Fold changes were found by normalizing each patient's values following ibrutinib treatment relative to the pre-treatment value.

Analysis by t-tests using linear mixed models with patient random effects to account for repeated measures across time points were used to test for changes in miR-155 expression. All tests were 2-sided and considered statistically significant when using a conservative Bonferroni correction within each analysis to control the overall family-wise type I error rate at α=0.05.

### DISCUSSION

Pre-treatment baseline miR-155 expression was measured in 109 patients for whom baseline samples were available that had been previously treated on two chemoimmunotherapy trials. Nanostring analysis showed the expression of miR-155 was above the background threshold in all patients. Patients were dichotomized as high (n=53) and low expressers (n=56) using the median value of miR-155 expression (median intensity=1154; range: 110-3265). The expression of miR-155 was not significantly associated with the majority of baseline demographic, clinical and cytogenetic characteristics, including age, Rai stage and high-risk cytogenetics del(17p)/del(11q) (all p>0.15). However, high miR-155 expression was significantly associated with IGHV un-mutated disease (p=0.03) and ZAP70 methylation <20% (p<0.001). Among the high miR-155 expressers, 81% and 94% had IGHV un-mutated disease and ZAP70 methylation, respectively, compared with 58% and 65% of low miR-155 expressers.

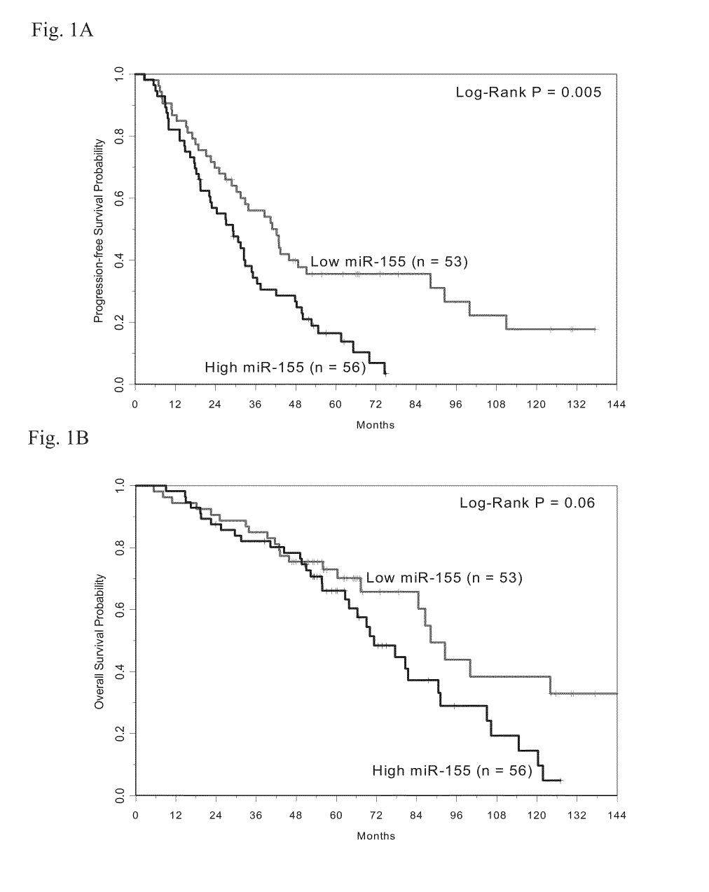

With respect to clinical outcome, patients with high miR-155 expression had a significantly shorter progression free survival (PFS) (p=0.005) and tend toward shorter overall survival (OS) (p=0.06) compared to those with low miR-155 expression (FIGS. 1A and 1B). The estimated median PFS and OS for high miR-155 expressers were 29 (95% CI: 20-35) and 71 months (95% CI: 63-91), respectively, versus 42 (95% CI: 29-51) and 88 months (95% CI: 67—not reached) for low miR-155 expressers. In a multivariable model for PFS, high miR-155 remained significantly associated with higher risk of relapse or death (HR=1.82, 95% CI: 1.13-2.94, p=0.01) when adjusting for high-risk cytogenetics and increased WBC. For OS, there was evidence of non-proportional hazards, where the risk of death increased with longer follow-up. In a model adjusting for hemoglobin, the risk of death in the first 4 years on study was not significantly different according to miR-155 expression (HR=0.95, 95% CI: 0.41-2.19, p=0.91), but thereafter, higher miR-155 expression was associated with increased risk of death (HR=3.25, 95% CI: 1.46-7.21, p=0.004).

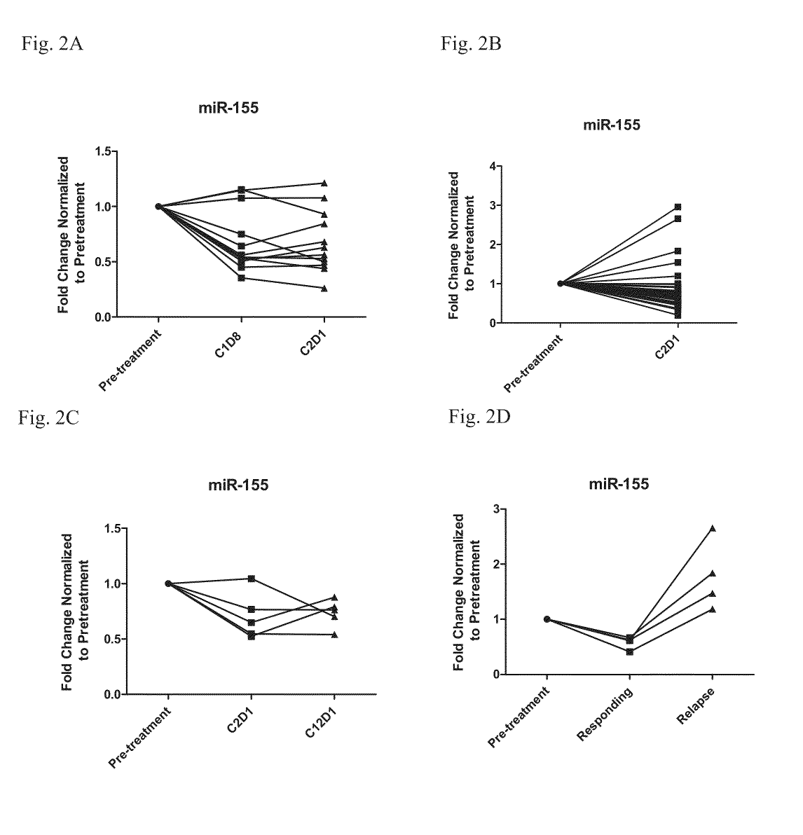

The regulation of BCR pathways through ibrutinib inhibition of BTK and its ability to modulate miR-155 was investigated. Initially blood samples from 12 CLL patients prior to receiving ibrutinib, after 1 week (C1D8) and after 29 days (C2D1) with treatment on OSU-10053 were examined. The miR-155 expression, assessed by quantitative real time PCR, was found significantly down-regulated at C1D8 and C2D1 relative to baseline (FIG. 2A). To confirm this, lymphocytes from 34 additional patients treated with ibrutinib on OSU-11133 were examined, and it was found that miR-155 expression post-treatment with ibrutinib was 0.71 times the expression prior to therapy (95% CI: 0.59-0.85, p=0.0006; FIG. 2B). Further the miR-155 expression was down-regulated at C2D1 in 29 (85%) of the patients studied.

The response pattern observed with ibrutinib includes traditional partial and complete responses, but also patients who have dramatic node disease reduction but persistent blood lymphocytosis that remains asymptomatic for an extended period of time without evidence of active proliferation. In contrast, patients who relapse after responding to ibrutinib typically have proliferative disease. Expression of miR-155 was measured in serial samples from patients with a partial response with persistent lymphocytosis at 1 year as well as in patients responding to ibrutinib with subsequent progressions to determine if expression patterns were similar or different. In patients with lymphocytosis, miR-155 expression decreased with 29 days of ibrutinib treatment and remained at this lower expression level at 1 year relative to baseline (p=0.013; FIG. 2C). In contrast, patients who relapsed with ibrutinib treatment showed elevated miR-155 expression relative to baseline (p=0.002; FIG. 2D), despite an initial decrease in expression with response.

The examples and embodiments described herein are for illustrative purposes only and various modifications or changes suggested to persons skilled in the art are to be included within the spirit and purview of this application and scope of the appended claims.

What is claimed is:

1. A method of assessing whether an individual having chronic lymphocytic leukemia (CLL) is responsive or likely to be responsive to therapy with ibrutinib, comprising:
a. administering a treatment comprising ibrutinib;
b. determining an expression level of miR-155 in a sample from the individual following administration of the treatment; and
c. characterizing the individual as responsive or likely to be responsive to therapy if the individual shows a decrease in the expression level of miR-155 relative to a control.

2. The method of claim 1, wherein the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib.

3. The method of claim 1, wherein the control is the expression level of miR-155 in the individual prior to treatment with ibrutinib.

4. The method of claim 1, wherein the expression level of miR-155 is measured on day 1, 2, 3, 4, 5, 6, 7, 8, 9, 10, 12, 14, 16, 18, 20, 25, 29, or more following treatment with ibrutinib.

5. The method of claim 1, wherein CLL is characterized by cytogenetic abnormalities.

6. The method of claim 5, wherein the cytogenetic abnormalities comprise del(17p13.1), del(11q22.3), del(11q23), unmutated IgVH together with ZAP-70+ and/or CD38+, trisomy 12, del(13q14), +(12q21), del(6q21), ATM del, p53 del, complex karyotype, or a combination thereof.

7. The method of claim 1, wherein CLL is a refractory CLL.

8. The method of claim 1, wherein CLL is a relapsed CLL.

9. The method of claim 1, wherein the sample is a blood sample or a serum sample.

10. The method of claim 1, wherein determining the expression level of miR-155 in the sample comprises measuring the amount of nucleic acid encoding miR-155 in the sample.

11. The method of claim 10, wherein the sample comprises one or more tumor cells.

12. The method of claim 1, wherein the treatment further comprises a second anticancer therapy.

13. The method of claim 12, wherein the second anticancer therapy is a chemotherapeutic agent.

14. The method of claim 13, wherein the chemotherapeutic agent is selected from among ofatumumab, rituximab, fludarabine, or a combination thereof.

15. The method of claim 14, wherein the chemotherapeutic agent is ofatumumab.

16. The method of claim 1, wherein the individual has received previous anticancer therapy.

17. The method of claim 1, wherein the individual has not received previous anticancer therapy.

18. The method of claim 1, wherein ibrutinib is administered at a dosage of about 40 mg/day to about 1000 mg/day.

19. A method of treating an individual having chronic lymphocytic leukemia (CLL), comprising:
a. administering a treatment comprising ibrutinib;
b. determining an expression level of miR-155 in a sample from the individual following administration of the treatment; and
c. continuing the treatment if the expression level of miR-155 is decreased by a predetermined amount relative to the expression level of miR-155 prior to the treatment.

20. A method of treating an individual having chronic lymphocytic leukemia (CLL), comprising:
a. administering a treatment comprising ibrutinib;
b. determining an expression level of miR-155 in a sample from the individual following administration of the treatment; and
c. discontinuing the treatment if the expression level of miR-155 is not decreased by a predetermined amount relative to the expression level of miR-155 prior to the treatment.

21. The method of claim 19, wherein the expression level of miR-155 decreases by 5%, 10%, 15%, 20%, 25%, 30%, 35%, 40%, 45%, 50%, 55%, 60%, 65%, 70%, 75%, 80%, 85%, 90%, 95%, 99% or greater following treatment with ibrutinib.
